(19) United States
(12) Patent Application Publication
(10) Pub. No.: US 2025/0074989 A1
(43) Pub. Date: Mar. 6, 2025

(54) METHODS AND COMPOSITIONS FOR TREATING PANCREATIC DISEASE

(71) Applicant: METANOIA BIO INC., Beverly Hills, CA (US)

(72) Inventors: Slavica TUDZAROVA-TRAJKOVSKA, Beverly Hills, CA (US); Oppel GREEFF, Beverly Hills, CA (US); Abraham VAN WYK, Beverly Hills, CA (US)

(21) Appl. No.: 18/561,199

(22) PCT Filed: May 16, 2022
(86) PCT No.: PCT/US2022/029388

§ 371 Date: Nov. 15, 2023

Related U.S. Application Data
(60) Provisional application No. 63/189,207, filed on May 16, 2021.

(51) Int. Cl. C07K 16/28; A61K 31/205; A61K 31/26; A61K 31/357; A61K 31/416; A61K 31/4196; A61K 31/4709; A61K 31/496; A61K 31/661; C12N 15/113

(52) U.S. Cl. CPC C07K 16/2857; A61K 31/205; A61K 31/26; A61K 31/357; A61K 31/416; A61K 31/4196; A61K 31/4709; A61K 31/496; A61K 31/661; C12N 15/1138; C07K 2317/569; C12N 2310/11

(57) ABSTRACT

The disclosure provides methods and compositions for treating pancreatic and hepatic disease such as, pancreatic steatosis, pancreatic cancer, non-alcoholic fatty liver disease, non-alcoholic steatohepatitis, and hepatic cancer. The provided methods include administering an effective amount of a HIF1-α Pathway Inhibitor or a HIF1-α Inhibitor and an PFKFB3 inhibitor to the subject having or at risk of having pancreatic and/or hepatic disease. The provided methods include administering an effective amount of a HIF1-α Pathway Inhibitor or a HIF1-α Inhibitor and an PFKFB3 inhibitor to the subject having or at risk of having pancreatic and/or hepatic disease.

### BACKGROUND

The disclosure provides methods and compositions for treating pancreatic and hepatic disease such as, pancreatic steatosis, pancreatic cancer, non-alcoholic fatty liver disease, non-alcoholic steatohepatitis, and hepatic cancer. The provided methods include administering an effective amount of a HIF1-α Pathway Inhibitor or a HIF1-α Inhibitor and an PFKFB3 inhibitor to the subject having or at risk of having pancreatic and/or hepatic disease.

Obesity and metabolic syndrome (including obesity, hyperglycemia, dyslipidemia, hypertension and insulin resistance) lead to metabolic derangements that result in lipid mishandling by adipocytes and fat infiltration in the pancreas and liver. Adipocytokine imbalances in circulation and in the microenvironments of the pancreas and liver cause chronic, low-grade inflammation and result in pancreatic and hepatic dysfunction. Furthermore, these adipocytokines regulate cell growth, differentiation, as well as angiogenesis and lymphatic spread.

Pancreatic steatosis describes a disease ranging from infiltration of fat in the pancreas to pancreatic inflammation, and the development of pancreatic fibrosis. The consequences of adipocyte infiltration in the pancreas lead to pancreatic dysfunction and are thought to initiate carcinogenesis, leading to pancreatic intraepithelial neoplasia and pancreatic ductal adenocarcinoma (PDAC).

Nonalcoholic fatty liver disease (NAFLD) refers to the presence of hepatic accumulation of triglycerides in the hepatocytes and is the most common cause of chronic liver disease in the Western world. Its clinical-histologic phenotype extends from nonalcoholic fatty liver (NAFL) to nonalcoholic steatohepatitis (NASH), characterized by liver inflammation and progressive fibrosis, leading to cirrhosis and end stage liver disease as well as hepatocellular carcinoma. NASH-related cirrhosis has become a leading indication for liver transplantation in the United States.

Type 2 diabetes (T2D) is a well-established risk factor of pancreatic adenocarcinoma (PDAC), increasing the risk of malignancy 1.5-2 fold, even after adjusting for correlating anthropometric factors like BMI. Preneoplastic pancreatic intraepithelial lesions (PanINs) occur more frequently and the replication of pancreatic duct glands (PDG) is increased 2-fold in pancreata from individuals with T2D when compared with BMI- and age-matched controls. In addition to acinar-to-ductal metaplasia, the PDG compartment is implicated in PDAC as an initiation niche for tumor development and may respond to changes in the pancreatic microenvironment (inflammation and metabolism) by increased proliferation, purportedly in attempt to regenerate ductal epithelium after injury e.g., during pancreatitis. Chronic activation of PDGs may result in overarching tumor promotion in PDGs or progression of PanINs. Moreover, pancreatic islet-PanIN complexes are enriched in pancreases from brain-dead donors with T2D compared to non-diabetics, indicating that diabetic islets in T2D can exert a profound paracrine effect on PanINs, given their anatomical proximity to PanINs.

The therapies available for treating pancreatic and hepatic diseases such as pancreatic cancer, hepatic cancer, pancreatitis and nonalcoholic fatty liver disease are limited and have associated drawbacks. Given the ever-increasing number of individuals afflicted with pancreatic and hepatic disease and their impact on a global scale, treatment methods that reduce or alleviate pancreatic and hepatic disease are urgently needed.

### BRIEF SUMMARY

The disclosure provides methods and compositions for treating pancreatic and hepatic disease such as, pancreatic steatosis, pancreatic cancer such as pancreatic duct adrenal carcinoma, non-alcoholic fatty liver, non-alcoholic steatohepatitis, and hepatic cancer such as hepatocellular carcinoma. The provided methods include administering an effective amount of a HIF1-α Pathway Inhibitor or a HIF1-α Inhibitor and an PFKFB3 inhibitor to the subject having or at risk of having pancreatic and/or hepatic disease.

In T2D, exocrine pancreas undergoes various changes including exocrine insufficiency, acinar fibrosis and acinar inflammation and the activation of the HIF-1α/PFKFB3 signaling axis in diabetic β-cells that lead to β-cell dysfunction, inflammation, pancreatic islet metabolic remodeling and lactate release-factors that can affect the microenvironment and provide tumour promotion cues in the exocrine pancreas.

The inventors have surprisingly discovered that the combination of an HIF1-α inhibitor and a PFKFB3 inhibitor of the HIF1-α/PFKFB3 signaling axis is able to treat pancreatic cancer by mitigating the events associated with the promotion or progression of pancreatic and hepatic cancer. Without being limited by theory, it is believed that the disclosed methods activate cell competition in the pancreas and liver that promotes the survival of functional cells, and thereby mitigates factors such as cell dysfunction, inflammation, metabolic remodeling and lactate release that promote the survival and dysfunctional cells and results in pancreatic and hepatic disease and tumor growth and progression.

The inventors have also discovered that lactate secreted from diabetic islets in the exocrine pancreas activates GPR81 signaling on proximal acinar cells, pancreatic ductal glands (PDGs) and in preexistent PanINs. Chronic HIF1-α-PFKFB3-dependent release of lactate exerts a tumor promoting effect on the exocrine pancreas, thereby propagating tumor growth and progression. GPR81 receptor is overexpressed in 98% of all cell lines derived from PDAC patients. GPR81 activation has been linked to the accumulation of intracellular lipid after inhibition of lipolysis, increased proliferation, migration, reactive oxygen species and (ROS) detoxification, multiple events leading to propagation of preneoplastic lesions in the pancreas.

In some embodiments, the provided methods and compositions treat a chronic pancreatic disease and/or hepatic disease. In some embodiments, the provided methods and compositions treat an acute pancreatic disease and/or hepatic disease.

In some embodiments, the disclosure provides:
[1] a method of treating a pancreatic steatosis in a subject in need thereof comprising:
   (a) administering an effective amount of a HIF1-α Pathway Inhibitor and an PFKFB3 inhibitor to the subject;
   (b) administering an effective amount of a HIF1-α Pathway Inhibitor to the subject, wherein the subject has previously been administered a PFKFB3 Inhibitor; or
   (c) administering an effective amount of a PFKFB3 Inhibitor to the subject, wherein the subject has previously been administered a HIF1-α Pathway Inhibitor; and wherein
   the PFKFB3 inhibitor does not inhibit PI3K/AKT/mTOR pathway or HIF1-α.
[2] the method of [1], wherein the subject is administered an effective amount of the HIF1-α Pathway Inhibitor and the PFKFB3 inhibitor;
[3] the method of [1], wherein the subject is administered an effective amount of the HIF1-α Pathway Inhibitor and wherein the subject has previously been administered the PFKFB3 Inhibitor;
[4] the method of [1], wherein the subject is administered an effective amount of the PFKFB3 Inhibitor and wherein the subject has previously been administered the HIF1-α Pathway Inhibitor;
[5] the method of any one of [1]-[4], wherein the method of any one of 1(a)-1(c) is administered as a prophylactic treatment for the pancreatic steatosis (e.g., infiltration of fat in the pancreas, infiltration of fat in the pancreas with pancreatic inflammation, and infiltration of fat in the pancreas with development of pancreatic fibrosis);
[6] the method of any one of [1]-[4], wherein the subject has or is at risk of having the pancreatic steatosis (e.g., infiltration of fat in the pancreas, infiltration of fat in the pancreas with pancreatic inflammation, and infiltration of fat in the pancreas with development of pancreatic fibrosis);
[7] the method of any one of [1]-[4], wherein the subject has or has been diagnosed as having the pancreatic steatosis;
[8] the method of any one of [1]-[7], wherein the pancreatic steatosis is associated with infiltration of fat in the pancreas, infiltration of fat in the pancreas with pancreatic inflammation, or infiltration of fat in the pancreas with development of pancreatic fibrosis;
[9] the method of any one of [1]-[8], wherein the pancreatic steatosis is associated with infiltration of fat in the pancreas;
[10] the method of any one of [1]-[8], wherein the pancreatic steatosis is associated with infiltration of fat in the pancreas with pancreatic inflammation;
[11] the method of any one of [1]-[8], wherein the pancreatic steatosis is associated with infiltration of fat in the pancreas with development of pancreatic fibrosis;
[12] the method of any one of [1]-[8], wherein the pancreatic steatosis is associated with infiltration of fat in the pancreas with pancreatic inflammation with development of pancreatic fibrosis;
[13] the method of any one of [1]-[12], wherein the administered HIF1-α Pathway Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, MiRNA, dsRNA, ssRNA, and shRNA), a peptibody, a nanobody, a HIF1-α Pathway binding polypeptide, or a small molecule HIF1-α Pathway Inhibitor;
[14] the method of any one of [1]-[13], wherein the administered HIF1-α Pathway Inhibitor is silibinin, PX-478 or YC-1, or a salt thereof;
[15] the method of any one of [1]-[14], wherein the administered HIF1-α Pathway Inhibitor is ganetespib (ST-9090), phenethyl isothocyanate, or BAY-87-2243, or a salt thereof;
[16] the method of any one of [1]-[15], wherein the administered HIF1-α Pathway Inhibitor is a HIF1-α Inhibitor;
[17] the method of [16], wherein the HIF1-α Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α binding polypeptide, or a small molecule HIF1-α Inhibitor;
[18] the method of or [17], wherein the administered HIF1-α Inhibitor is antisense oligonucleotide EZN-2968 or nanobody AG-1, AG-2, AG-3, AG-4, AG-5, VHH212, or AHPC;
[19] the method of any one of [1]-[18], wherein the administered PFKFB3 Inhibitor is an antibody or antigen-binding antibody fragment (e.g., a single chain antibody, a single-domain antibody, a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor;
[20] the method of any one of [1]-[19], wherein the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof;
[21] the method of any one of [1]-[20], wherein the administered PFKFB3 Inhibitor: (a) is KAN0436151 or KAN0436067, or a salt thereof; (b) has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof; (c) has the structure of formula AZ44-AZ70 or AZ71, depicted in FIG. 1E, or a salt thereof; or (d) is AZ67, or a salt thereof;
[22] the method of any one of [1]-[21], wherein the HIF1-α Pathway Inhibitor and the PFKFB3 inhibitor are co-administered to the subject;
[23] the method of any one of [1]-[22], wherein the administration of the HIF1-α Pathway Inhibitor and/or the PFKFB3 inhibitor administration is oral, parenteral, orthotopic, intradermal, subcutaneous, intramuscular, intraperitoneal, intranasal, intratumoral, or intravenous;
[24] the method of any one of [1]-[23], wherein the HIF1-α Pathway Inhibitor and the PFKFB3 inhibitor are administered before the onset of one or more symptoms of the pancreatic steatosis;
[25] the method of any one of [1]-[24], wherein treating the pancreatic steatosis comprises delaying the onset of the pancreatic steatosis;
[26] the method of any one of [1]-[23], wherein the HIF1-α Pathway Inhibitor and the PFKFB3 inhibitor are administered after the onset of one or more symptoms of the pancreatic steatosis;
[27] the method of [26], wherein the method results in one or more symptoms of the pancreatic steatosis are reduced in the subject administered the HIF1-α Pathway Inhibitor and the PFKFB3 inhibitor compared to in the subject prior to treatment;
[28] the method of [27], wherein the one or more reduced symptoms of the pancreatic steatosis is indicated by: a reduced fat content in the pancreas to less than 25%, reduced abdominal pain; reduced nausea, increased appetite, weight gain, reduced jaundice, reduced edema; and reduced tiredness, mental confusion, or weakness, reduced inflammation of the pancreas, reduced pancreatitis, reduced fibrosis in cells of the pancreas, and reduced levels of a pancreatic steatosis biomarker or pro-inflammatory cytokine (e.g., TNFα, IL-1β, IL6, MCP1, IL8, PAF);
[29] the method of or [28], wherein the one or more of the reduced symptoms is the reduction of fat content in the subject by at least 10%, at least 20%, at least 30%, at least 40%, or at least 50% compared to in the subject prior to treatment with the HIF1-α Pathway Inhibitor and the PFKFB3 inhibitor;
[30] the method according to any one of [27]-[29], wherein the serum levels of at least 1, 2, 3, 4, or 5 pancreatic steatosis biomarkers is reduced by at least 20%, compared to in the subject prior to treatment with the HIF1-α Pathway Inhibitor and the PFKFB3 inhibitor;
[31] the method according to any one of [27]-[30], wherein the serum levels of at least 1, 2, 3, 4, or 5 of the biomarkers: TNFα, IL-1β, IL6, MCP1, IL8, and/or PAF are reduced by at least 20%, compared to in the subject prior to treatment with the HIF1-α Pathway Inhibitor and the PFKFB3 inhibitor;
[32] the method of any one of [1]-[31], which further comprises administering an additional therapeutic agent to the subject;
[33] a method of treating a pancreatic cancer in a subject in need thereof comprising:
   (a) administering an effective amount of a HIF1-α Pathway Inhibitor and an PFKFB3 inhibitor to the subject;
   (b) administering an effective amount of a HIF1-α Pathway Inhibitor to the subject, wherein the subject has previously been administered a PFKFB3 Inhibitor; or
   (c) administering an effective amount of a PFKFB3 Inhibitor to the subject, wherein the subject has previously been administered a HIF1-α Pathway Inhibitor; and
   wherein the PFKFB3 inhibitor does not inhibit PI3K/AKT/mTOR pathway or HIF1-α;
[34] the method of [33], wherein the subject is administered an effective amount of the HIF1-α Pathway Inhibitor and the PFKFB3 inhibitor;
[35] the method of [33], wherein the subject is administered an effective amount of the HIF1-α Pathway Inhibitor and wherein the subject has previously been administered the PFKFB3 Inhibitor;
[36] the method of [33], wherein the subject is administered an effective amount of the PFKFB3 Inhibitor and wherein the subject has previously been administered the HIF1-α Pathway Inhibitor;
[37] the method of any one of [33]-[36], wherein the method of any one of 1(a)-1(c) is administered as a prophylactic treatment for the pancreatic cancer;
[38] the method of any one of [33]-[36], wherein the subject has or is at risk of having the pancreatic cancer;
[39] the method of any one of [33]-[36], wherein the subject has or has been diagnosed as having the pancreatic cancer;
[40] the method of any one of [33]-[39], wherein the pancreatic cancer is an exocrine pancreatic cancer or a neuroendocrine pancreatic cancer;
[41] the method of [40], wherein the pancreatic cancer is an exocrine pancreatic cancer (e.g., an adenocarcinoma, squamous cell carcinoma, adenosquamous carcinoma, or a colloid carcinoma);
[42] the method of [40], wherein the pancreatic cancer is a ductal adenocarcinoma (PDAC);
[43] the method of [40], wherein the pancreatic cancer is a squamous cell carcinoma, adenosquamous carcinoma, or a colloid carcinoma;
[44] the method of [40], wherein the pancreatic cancer is a neuroendocrine pancreatic cancer;
[45] the method of any one of [33]-[44], wherein the administered HIF1-α Pathway Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, dsRNA, ssRNA, and shRNA), a peptibody, a nanobody, a HIF1-α Pathway binding polypeptide, or a small molecule HIF1-α Pathway Inhibitor;
[46] the method of any one of [33]-[45], wherein the administered HIF1-α Pathway Inhibitor is silibinin, PX-478 or YC-1, or a salt thereof;
[47] the method of any one of [33]-[45], wherein the administered HIF1-α Pathway Inhibitor is ganetespib (ST-9090), phenethyl isothocyanate, or BAY-87-2243, or a salt thereof;
[48] the method of any one of [33]-[47], wherein the administered HIF1-α Pathway Inhibitor is a HIF1-α Inhibitor;
[49] the method of [48], wherein the HIF1-α Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α binding polypeptide, or a small molecule HIF1-α
[50] the method of [48 or 49], wherein the administered HIF1-α Inhibitor is Antisense oligonucleotide EZN-2968 or nanobody AG-1, AG-2, AG-3, AG-4, AG-5, VHH212, or AHPC;
[51] the method of any one of [33]-[50], wherein the administered PFKFB3 Inhibitor is an antibody or antigen-binding antibody fragment (e.g., a single chain antibody, a single-domain antibody, a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor;
[52] the method of any one of [33]-[51], wherein the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof;
[53] the method of any one of [33]-[51], wherein the administered PFKFB3 Inhibitor: (a) is KAN0436151 or KAN0436067, or a salt thereof; (b) has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof; (c) has the structure of formula AZ44-AZ70 or AZ71, depicted in FIG. 1E, or a salt thereof; or (d) is AZ67, or a salt thereof
[54] the method of any one of [33]-[53], wherein the HIF1-α Pathway Inhibitor and the PFKFB3 inhibitor are co-administered to the subject;
[55] the method of any one of [33]-[54], wherein the administration of the HIF1-α Pathway Inhibitor and/or the PFKFB3 inhibitor administration is oral, parenteral, orthotopic, intradermal, subcutaneous, intramuscular, intraperitoneal, intranasal, intratumoral, or intravenous.
[56] the method of any one of [33]-[55], wherein the HIF1-α Pathway Inhibitor and the PFKFB3 inhibitor are administered before the onset of one or more symptoms of the pancreatic cancer.
[57] the method of any one of [33]-[56], wherein treating the pancreatic cancer comprises delaying the onset of the pancreatic cancer.
[58] the method of any one of [33]-[57], wherein the HIF1-α Pathway Inhibitor and the PFKFB3 inhibitor are administered after the onset of one or more symptoms of the pancreatic cancer.
[59] the method of any one of [33]-[58], wherein the method results in reduction in one or more symptoms of the pancreatic cancer in the subject compared to prior to administration of the HIF1-α Pathway Inhibitor and the PFKFB3 inhibitor.
[60] the method of [59], wherein the one or more reduced symptoms of the pancreatic cancer is indicated by: decrease in tumor size, a suppression or decrease in tumor growth, no new tumor formation, a decrease in new tumor formation, an increase in survival or progression-free survival, no metastases, an increase in treatment options, delay in time from surgery to recurrence, reduction in jaundice, suppression of spread to liver, reduction in pain, improved appetite, improved digestion, reduction of gallbladder size, and reduced incidence of blood clots;
[61] the method of or [60], wherein the one or more symptoms of the pancreatic cancer are reduced by at least 10%, at least 20%, at least 30%, at least 40%, or at least 50% compared to in the subject prior to treatment with the HIF1-α Pathway Inhibitor and the PFKFB3 inhibitor;
[62] the method according to any one of [59]-[61], wherein at least one of the biomarkers creatine kinase (CK-MB), troponin, N-terminal pro B-type natriuretic peptide, alpha-1 antitrypsin, C-reactive protein, apolipoprotein A1, apolipoprotein B, creatinine, alkaline phosphatase, and transferrin is reduced compared to in the subject prior to treatment with the HIF1-α Pathway Inhibitor and the PFKFB3 inhibitor;
[63] the method according to any one of [59]-[62], wherein
   (a) at least 1, 2, 3, 4, 5 or more of the biomarkers, creatine kinase (CK-MB), troponin, N-terminal pro B-type natriuretic peptide, alpha-1 antitrypsin, C-reactive protein, apolipoprotein A1, apolipoprotein B, creatinine, alkaline phosphatase, and transferrin of the subject are improved by at least 20%, at least 30%, at least 40%, or at least 50% compared to in the subject prior to treatment with the HIF1-α Pathway Inhibitor and the PFKFB3 inhibitor; or
   (b) the tumor size in the subject is reduced by at least 20%, at least 30%, at least 40%, or at least 50% compared to in the subject prior to treatment with the HIF1-α Pathway Inhibitor and the PFKFB3 inhibitor
[64] the method of any one of [33]-[63], which further comprises administering an additional therapeutic agent to the subject.
[65] a method of treating non-alcoholic fatty liver disease (NAFLD) in a subject in need thereof comprising:
   (a) administering an effective amount of a HIF1-α Pathway Inhibitor and an PFKFB3 inhibitor to the subject;
   (b) administering an effective amount of a HIF1-α Pathway Inhibitor to the subject, wherein the subject has previously been administered a PFKFB3 Inhibitor; or
   (c) administering an effective amount of a PFKFB3 Inhibitor to the subject, wherein the subject has previously been administered a HIF1-α Pathway Inhibitor; and
   wherein the PFKFB3 inhibitor does not inhibit PI3K/AKT/mTOR pathway or HIF1-α.
[66] the method of [65], wherein the subject is administered an effective amount of the HIF1-α Pathway Inhibitor and the PFKFB3 inhibitor;
[67] the method of [65], wherein the subject is administered an effective amount of the HIF1-α Pathway Inhibitor and wherein the subject has previously been administered the PFKFB3 Inhibitor;
[68] the method of [65], wherein the subject is administered an effective amount of the PFKFB3 Inhibitor and wherein the subject has previously been administered the HIF1-α Pathway Inhibitor;
[69] the method of any one of [65]-[68], wherein the method of any one of 1(a)-1(c) is administered as a prophylactic treatment for non-alcoholic fatty liver disease (e.g., non-alcoholic fatty liver (NAFL), non-alcoholic steatohepatitis (NASH), or NAFLD-associated liver fibrosis);
[70] the method of any one of [65]-[68], wherein the subject has or is at risk of having non-alcoholic fatty liver disease (e.g., non-alcoholic fatty liver (NAFL), non-alcoholic steatohepatitis (NASH), or NAFLD-associated liver fibrosis);
[71] the method of any one of [65]-[68], wherein the subject has or has been diagnosed as having non-alcoholic fatty liver disease (e.g., non-alcoholic fatty liver (NAFL), non-alcoholic steatohepatitis (NASH), or NAFLD-associated liver fibrosis);
[72] the method of any one of [65]-[71], wherein the administered HIF1-α Pathway Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, dsRNA, ssRNA, and shRNA), a peptibody, a nanobody, a HIF1-α Pathway binding polypeptide, or a small molecule HIF1-α Pathway Inhibitor;
[73] the method of any one of [65]-[72], wherein the administered HIF1-α Pathway Inhibitor is silibinin, PX-478 or YC-1, or a salt thereof;
[74] the method of any one of [65]-[72], wherein the administered HIF1-α Pathway Inhibitor is ganetespib (ST-9090), phenethyl isothocyanate, or BAY-87-2243, or a salt thereof;
[75] the method of any one of [65]-[72], wherein the administered HIF1-α Pathway Inhibitor is a HIF1-α Inhibitor.
[76] the method of [75], wherein the HIF1-α Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α binding polypeptide, or a small molecule HIF1-α
[77] the method of or [76], wherein the administered HIF1-α Inhibitor is Antisense oligonucleotide EZN-2968 or nanobody AG-1, AG-2, AG-3, AG-4, AG-5, VHH212, or AHPC;
[78] the method of any one of [65]-[77], wherein the administered PFKFB3 Inhibitor is an antibody or antigen-binding antibody fragment (e.g., a single chain antibody, a single-domain antibody, a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor;
[79] the method of any one of [65]-[78], wherein the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof;
[80] the method of any one of [65]-[78], wherein the administered PFKFB3 Inhibitor; (a) is KAN0436151 or KAN0436067, or a salt thereof; (b) has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof; (c) has the structure of formula AZ44-AZ70 or AZ71, depicted in FIG. 1E, or a salt thereof; or (d) is AZ67, or a salt thereof:
[81] the method of any one of [65]-[80], wherein the HIF1-α Pathway Inhibitor and the PFKFB3 inhibitor are co-administered to the subject;
[82] the method of any one of [65]-[81], wherein the administration of the HIF1-α Pathway Inhibitor and/or the PFKFB3 inhibitor administration is oral, parenteral, orthotopic, intradermal, subcutaneous, intramuscular, intraperitoneal, intranasal, intratumoral, or intravenous.
[83] the method of any one of [65]-[82], wherein the HIF1-α Pathway Inhibitor and the PFKFB3 inhibitor are administered before the onset of one or more symptoms of non-alcoholic fatty liver disease (e.g., non-alcoholic fatty liver (NAFL), non-alcoholic steatohepatitis (NASH), or NAFLD-associated liver fibrosis);
[84] the method of any one of [65]-[83], wherein treating non-alcoholic fatty liver disease (NAFLD) comprises delaying the onset of non-alcoholic fatty liver disease (e.g., non-alcoholic fatty liver (NAFL), non-alcoholic steatohepatitis (NASH), or NAFLD-associated liver fibrosis);
[85] the method of any one of [65]-[84], wherein the HIF1-α Pathway Inhibitor and the PFKFB3 inhibitor are administered after the onset of one or more symptoms of non-alcoholic fatty liver disease (e.g., non-alcoholic fatty liver (NAFL), non-alcoholic steatohepatitis (NASH), or NAFLD-associated liver fibrosis);
[86] the method of any one of [65]-[85], wherein the method results in reduction in one or more symptoms of non-alcoholic fatty liver disease in the subject compared to prior to administration of the HIF1-α Pathway Inhibitor and the PFKFB3 inhibitor.
[87] the method of [86], wherein the one or more reduced symptoms of non-alcoholic fatty liver disease is indicated by:
   (a) reduced fatigue, pain or discomfort in the upper right abdomen, reduction in spleen enlargement, reduced jaundice; reduced edema; increased appetite, and reduced;
   (b) reduced NAS; or
   (c) reduced levels reduced levels of NAFLD biomarkers (e.g., TNFα, IL-6, CRP, IL-1RA, PAI1, CXCL10, CK18, FGF21, oxLDL hyaluronic acid, laminin, procollagen II, and TIMP1);
[88] the method of or [87], wherein the one or more symptoms of non-alcoholic fatty liver disease are reduced by at least 10%, at least 20%, at least 30%, at least 40%, or at least 50% compared to in the subject prior to treatment with the HIF1-α Pathway Inhibitor and the PFKFB3 inhibitor;
[89] the method according to any one of [86]-[88], wherein
   (a) at least one of the plasma NAFLD biomarkers (e.g., TNFα, IL-6, CRP, IL-1RA, PAI1, CXCL10, CK18, FGF21, oxLDL, ALT, hyaluronic acid, laminin, procollagen II, and TIMP1) is improved compared to in the subject prior to treatment with the HIF1-α Pathway Inhibitor and the PFKFB3 inhibitor; or
   (b) the NAS score in the subject is reduced by 1, 2, 3, or more compared to in the subject prior to treatment with the HIF1-α Pathway Inhibitor and the PFKFB3 inhibitor, or
   (c) the treated subject has a NAS of <3 points;
[90] the method according to any one of [86]-[89], wherein at least 1, 2, 3, 4, or 5, wherein at least 1, 2, 3, 4, or 5, biomarkers selected from TNFα, IL-6, CRP, IL-1RA, PAI1, CXCL10, CK18, FGF21, oxLDL hyaluronic acid, laminin, procollagen II, and TIMP1 are reduced by at least 20%, at least 30%, at least 40%, or at least 50% compared to in the subject prior to treatment with the HIF1-α Pathway Inhibitor and the PFKFB3 inhibitor;
[91] the method of any one of [65]-[90], which further comprises administering an additional therapeutic agent to the subject;
[92] a method of treating hepatocellular carcinoma in a subject in need thereof comprising:
   (a) administering an effective amount of a HIF1-α Pathway Inhibitor and an PFKFB3 inhibitor to the subject;
   (b) administering an effective amount of a HIF1-α Pathway Inhibitor to the subject, wherein the subject has previously been administered a PFKFB3 Inhibitor; or
   (c) administering an effective amount of a PFKFB3 Inhibitor to the subject, wherein the subject has previously been administered a HIF1-α Pathway Inhibitor; and
   wherein the PFKFB3 inhibitor does not inhibit PI3K/AKT/mTOR pathway or HIF1-α;
[93] the method of [92], wherein the subject is administered an effective amount of the HIF1-α Pathway Inhibitor and the PFKFB3 inhibitor;
[94] the method of [92], wherein the subject is administered an effective amount of the HIF1-α Pathway Inhibitor and wherein the subject has previously been administered the PFKFB3 Inhibitor;
[95] the method of [92], wherein the subject is administered an effective amount of the PFKFB3 Inhibitor and wherein the subject has previously been administered the HIF1-α Pathway Inhibitor;
[96] the method of any one of [92]-[95], wherein the method of any one of [92 (a)-(c)] is administered as a prophylactic treatment for hepatocellular carcinoma;
[97] the method of any one of [92]-[95], wherein the subject has or is at risk of having hepatocellular carcinoma;
[98] the method of any one of [92]-[95], wherein the subject has or has been diagnosed as having hepatocellular carcinoma;
[99] the method of any one of [92]-[98], wherein the administered HIF1-α Pathway Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, dsRNA, ssRNA, and shRNA), a peptibody, a nanobody, a HIF1-α Pathway binding polypeptide, or a small molecule HIF1-α Pathway Inhibitor;
[100] the method of any one of [92]-[99], wherein the administered HIF1-α Pathway Inhibitor is silibinin, PX-478 or YC-1, or a salt thereof;
[101] the method of any one of [92]-[99], wherein the administered HIF1-α Pathway Inhibitor is ganetespib (ST-9090), phenethyl isothocyanate, or BAY-87-2243, or a salt thereof;
[102] the method of any one of [92]-[99], wherein the administered HIF1-α Pathway Inhibitor is a HIF1-α Inhibitor;
[103] the method of [102], wherein the HIF1-α Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α binding polypeptide, or a small molecule HIF1-α Inhibitor;
[104] the method of or [103], wherein the administered HIF1-α Inhibitor is Antisense oligonucleotide EZN-2968 or nanobody AG-1, AG-2, AG-3, AG-4, AG-5, VHH212, or AHPC;
[105] the method of any one of [92]-[104], wherein the administered PFKFB3 Inhibitor is an antibody or antigen-binding antibody fragment (e.g., a single chain antibody, a single-domain antibody, a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor;
[106] the method of any one of [92]-[105], wherein the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof;
[107] the method of any one of [92]-[105], wherein the administered PFKFB3 Inhibitor is: (a) is KAN0436151 or KAN0436067, or a salt thereof; (b) has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof; (c) has the structure of formula AZ44-AZ70 or AZ71, depicted in FIG. 1E, or a salt thereof; or (d) is AZ67, or a salt thereof;
[108] the method of any one of [92]-[107], wherein the HIF1-α Pathway Inhibitor and the PFKFB3 inhibitor are co-administered to the subject;
[109] the method of any one of [92]-[108], wherein the administration of the HIF1-α Pathway Inhibitor and/or the PFKFB3 inhibitor administration is oral, parenteral, orthotopic, intradermal, subcutaneous, intramuscular, intraperitoneal, intranasal, intratumoral, or intravenous;
[110] the method of any one of [92]-[109], wherein the HIF1-α Pathway Inhibitor and the PFKFB3 inhibitor are administered before the onset of one or more symptoms of hepatocellular carcinoma;
[111] the method of any one of [92]-[110], wherein treating hepatocellular carcinoma comprises delaying the onset of hepatocellular carcinoma;
[112] the method of any one of [92]-[111], wherein the HIF1-α Pathway Inhibitor and the PFKFB3 inhibitor are administered after the onset of one or more symptoms of hepatocellular carcinoma;
[113] the method of any one of [92]-[112], wherein the method results in reduction in one or more symptoms of hepatocellular carcinoma the subject administered the HIF1-α Pathway Inhibitor and the PFKFB3 inhibitor compared to in the subject prior to treatment;
[114] the method of [113], wherein the one or more reduced symptoms of hepatocellular carcinoma is indicated by:
   (a) weight gain, increased appetite, reduced fever, reduced nausea, reduced fatigue, reduced upper abdominal pain, reduced swelling in the abdomen and legs, reduced yellowing of the skin (jaundice), or reduced bruising;
   (b) reduction of at least one biomarker typically elevated in subjects having HCC (e.g., EpCam, VEGF, EGFR, FLT1, theophylline, HCC-22-5, KRT23, AHSG, FTL, C16Cer, C16DHC, C18DHC, SIP, C24DHC, C24:1DHC, C18Cer, C20Cer, C24Cer, C24:1Cer, Sphingosine, and SA1P, CTSD, HYOU1, PSAP, and LAMP-2); or
   (c) a decrease in tumor size, a suppression or decrease in tumor growth, no new tumor formation, a decrease in new tumor formation, an increase in survival or progression-free survival, no metastases, an increase in treatment options, or a delay in time from surgery to recurrence;
[115] the method of or [114], wherein at least one of the following symptoms is improved in the subject compared to prior to treatment with the HIF1-α Pathway Inhibitor and the PFKFB3 inhibitor: weight loss, loss of appetite, fever, nausea, fatigue, upper abdominal pain, swelling in the abdomen and legs, yellowing of the skin (jaundice), easy bruising or bleeding, and liver failure;
[116] the method according to any one of [113]-[115], wherein at least 1, 2, 3, 4, or 5 biomarkers selected from: EpCam, VEGF, EGFR, FLT1, theophylline, HCC-22-5, KRT23, AHSG, FTL, C16Cer, C16DHC, C18DHC, SIP, C24DHC, C24:1DHC, C18Cer, C20Cer, C24Cer, C24:1Cer, Sphingosine, and SA1P, CTSD, HYOU1, PSAP, and LAMP-2, is reduced at least 10%, at least 20%, at least 30%, at least 40%, or at least 50% compared to in the subject prior to treatment with the HIF1-α Pathway Inhibitor and the PFKFB3;
[117] the method according to any one of [113]-[116], wherein the tumor size in the subject is reduced by at least 20%, at least 30%, at least 40%, or at least 50% compared to in the subject prior to treatment with the HIF1-α Pathway Inhibitor and the PFKFB3 inhibitor;
[118] the method of any one of [92]-[117], which further comprises administering an additional therapeutic agent to the subject; and/or
[119] The method of any one of [1]-[118], which further comprises administering a GPR81 Inhibitor to the subject.

### BRIEF DESCRIPTION OF THE DRAWINGS/FIGURES

FIGS. 1A-1E, depict exemplary PFKFB3 small molecule inhibitors.

### DETAILED DESCRIPTION

### Definitions

Unless otherwise defined, all technical and scientific terms used herein have the same meaning as commonly understood by one of ordinary skill in the art to which this disclosure pertains. Although methods and materials similar or equivalent to those described herein can be used in the practice or testing of the provided compositions, suitable methods and materials are described below. Each publication, patent application, patent, and other reference mentioned herein is herein incorporated by reference in its entirety. In case of conflict, the present specification, including definitions, will control. In addition, the materials, methods, and examples are illustrative only and are not intended to be limiting.

Other features and advantages of the disclosed methods and compositions will be apparent from the following disclosure, drawings, and claims.

It is understood that wherever embodiments, are described herein with the language “comprising” otherwise analogous embodiments, described in terms of “containing” “consisting of” and/or “consisting essentially of” are also provided. However, when used in the claims as transitional phrases, each should be interpreted separately and in the appropriate legal and factual context (e.g., in claims, the transitional phrase “comprising” is considered more of an open-ended phrase while “consisting of” is more exclusive and “consisting essentially of” achieves a middle ground).

As used herein, the singular form “a”, “an”, and “the”, include plural forms unless it is expressly stated or is unambiguously clear from the context that such is not intended. The singular form “a”, “an”, and “the” also includes the statistical mean composition, characteristics, or size of the particles in a population of particles (e.g., mean polyethylene glycol molecular weight mean liposome diameter, mean liposome zeta potential). The mean particle size and zeta potential of liposomes in a pharmaceutical composition can routinely be measured using methods known in the art, such as dynamic light scattering. The mean amount of a therapeutic agent in a nanoparticle composition may routinely be measured for example, using absorption spectroscopy (e.g., ultraviolet-visible spectroscopy).

As used herein, the terms “approximately” and “about,” as applied to one or more values of interest, refer to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). For example, when used in the context of an amount of a given compound in a lipid component of a nanoparticle composition, “about” may mean +/−10% of the recited value. For instance, a nanoparticle composition including a lipid component having about 40% of a given compound may include 30-50% of the compound.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Recitation of ranges of values herein are merely intended to serve as a shorthand method of referring individually to each separate value falling within the range, unless otherwise indicated herein, and each separate value is incorporated into the specification as if it were individually recited herein.

Where embodiments of the disclosure are described in terms of a Markush group or other grouping of alternatives, the disclosed composition or method encompasses not only the entire group listed as a whole, but also each member of the group individually and all possible subgroups of the main group, and also the main group absent one or more of the group members. The disclosed methods and compositions also envisage the explicit exclusion of one or more of any of the group members in the disclosed compositions or methods.

The terms “antibody” and “antigen-binding antibody fragment” and the like, as used herein, include any protein or peptide containing molecule that comprises at least a portion of an immunoglobulin molecule, such as, but not limited to, at least one complementarity determining region (CDR) of a heavy or light chain or an antigen binding portion thereof.

The term “antibody” also includes fragments, specified portions and variants thereof, including antibody mimetics or comprising portions of antibodies that mimic the structure and/or function of an antibody or specified fragment or portion thereof, including single chain antibodies, single binding domain antibodies and antigen binding antibody fragments.

The term “antibody fragment” refers to a portion of an intact antibody, generally the antigen binding or variable region of an intact antibody. Examples of antibody fragments include, but are not limited to Fab, Fab′. F(ab′)2, single chain (scFv) and Fv fragments, diabodies; linear antibodies; single-chain antibody molecules; single Fab arm “one arm” antibodies and multispecific antibodies formed from antibody fragments, among others.

Antibody fragments include any protein or peptide containing molecule that comprises at least a portion of an immunoglobulin molecule, such as but not limited to, at least one complementarity determining region (CDR) of a heavy or light chain or a ligand binding portion thereof, a heavy chain or light chain variable region, a heavy chain or light chain constant region, a framework region, or any portion thereof, or at least one portion of an antigen or antigen receptor or binding protein, which can be incorporated into an antibody provided herein.

Antibody fragments can be produced by enzymatic cleavage, synthetic or recombinant techniques, as known in the art. Antibodies can also be produced in a variety of truncated forms using antibody genes in which one or more stop codons have been introduced upstream of the natural stop site. For example, a combination gene encoding a F(ab′)2 heavy chain portion can be designed to include DNA sequences encoding the CH1 domain and/or hinge region of the heavy chain. The various portions of antibodies can be joined together chemically by conventional techniques, or can be prepared as a contiguous protein using genetic engineering techniques.

The terms “nucleic acid” and “oligonucleotide” are used interchangeably herein and refer to at least two nucleotides covalently linked together. In some embodiments the HIF1-α pathway inhibitor and/or PFKFB3 inhibitor administered according to the provided methods is a therapeutic nucleic acid. In some embodiments, the administered nucleic acid is an ENMD-1198, an shRNA, a Dicer substrate (e.g., dsRNA), an miRNA, an anti-miRNA, an antisense molecule, a decoy, or an aptamer, or a plasmid capable of expressing a ENMD-1198, an shRNA, a Dicer substrate, an miRNA, an anti-miRNA, an antisense molecule, a decoy, or an aptamer.

The nucleic acids administered according to the provided methods are preferably single-stranded or double-stranded and generally contain phosphodiester bonds, although in some cases, nucleic acid/oligonucleotide analogs are included that have alternate backbones, comprising, for example, phosphoramide, phosphorathioate, phosphorodithioate, O-methylphosphoroamidiate linkages, and peptide nucleic acid backbones and linkages. Other analog nucleic acids/oligonucleotides include those with positive backbones; non-ionic backbones, and non-ribose backbones. Nucleic acids/oligonucleotides containing one or more carbocyclic sugars are also included within the definition of nucleic acids and oligonucleotides. These modifications of the ribose-phosphate backbone may be done for example, to facilitate the addition of additional moieties such as labels, or to increase the stability and half-life of such molecules in physiological environments. Nucleic acid/oligonucleotide backbones of oligonucleotides used according to the provided methods can range from about 5 nucleotides to about 750 nucleotides. Preferred nucleic acid/oligonucleotide backbones range from about 5 nucleotides to about 500 nucleotides, and preferably from about 10 nucleotides to about 100 nucleotides in length.

The oligonucleotides administered according to the provided methods are polymeric structures of nucleoside and/or nucleotide monomers capable of specifically hybridizing to at least a region of a nucleic acid target. As indicated above, the “nucleic acids” and “oligonucleotides” used according to the provided methods include, but are not limited to, compounds comprising naturally occurring bases, sugars and intersugar (backbone) linkages, non-naturally occurring modified monomers, or portions thereof (e.g., oligonucleotide analogs or mimetics) which function similarly to their naturally occurring counterpart, and combinations of these naturally occurring and non-naturally occurring monomers. As used herein, the term “modified” or “modification” includes any substitution and/or any change from a starting or natural oligomeric compound, such as an nucleic acid. Modifications to nucleic acids encompass substitutions or changes to internucleoside linkages, sugar moieties, or base moieties, such as those described herein and those otherwise known in the art.

As used herein, a “small molecule” refers to an organic compound that is either synthesized via conventional organic chemistry methods (e.g., in a laboratory) or found in nature. Typically, a small molecule is characterized in that it contains several carbon-carbon bonds, and has a molecular weight of less than about 1500 grams/mole. In certain embodiments, small molecules are less than about 1000 grams/mole. In certain embodiments, small molecules are less than about 550 grams/mole. In certain embodiments, small molecules are between about 200 and about 550 grams/mole. In certain embodiments, small molecules exclude peptides (e.g., compounds comprising 2 or more amino acids joined by a peptidyl bond). In certain embodiments, small molecules exclude nucleic acids.

The terms “condition” and “disease” are used interchangeably herein and refer to any condition or disorder that damages, interferes with or dysregulates the normal function of a cell, tissue, or organ.

“Fibrosis” is a pathological condition where fibrous connective tissue invades any organ (e.g., the pancreas and liver), usually as a consequence of inflammation or other injury.

As used herein, the term “pancreatic cancer” refers to the art recognized disease and includes cancers that originate in the tissue that comprises a pancreas. In various embodiments, the pancreatic cancer is an exocrine pancreatic cancer, a pancreatic cystic neoplasm or a pancreatic endocrine tumor. A subject who is “afflicted with pancreatic cancer”, or who is “diagnosed as having pancreatic cancer” is one who is clinically diagnosed with such a cancer by a qualified clinician, or one who exhibits one or more signs or symptoms (for example, reduced levels of a pancreatic cancer biomarker in gastrointestinal lavage fluid or fecal matter) of such a cancer and is subsequently clinically diagnosed with such a cancer by a qualified physician.

As used herein, the term “hepatocellular cancer” refers to the art recognized disease and includes cancers that originate in the tissue that comprises a liver. A subject who is “afflicted with hepatocellular cancer”, or who is “diagnosed as having hepatocellular cancer” is one who is clinically diagnosed with such a cancer by a qualified clinician, or one who exhibits one or more signs or symptoms (for example, reduced levels of a hepatocellular cancer biomarker of such a cancer and is subsequently clinically diagnosed with such a cancer by a qualified physician.

As used herein, the term pancreatic or hepatic cancer “biomarker” refers to a protein or non-proteinaceous substance which is differentially present in a biological sample (e.g., a gastrointestinal lavage fluid or fecal matter) in subjects afflicted with pancreatic or hepatic cancer as compared to subjects without pancreatic or hepatic cancer. In particular embodiments, the pancreatic cancer or hepatic cancer biomarker is a protein derived from the pancreas or liver, respectively. In other embodiments, the pancreatic cancer or hepatic cancer biomarker is a protein derived from non-pancreatic or non-hepatic sources, respectively (e.g., in the gastrointestinal tract, e.g., the intestine).

The “level” of pancreatic cancer or hepatic cancer biomarker, as used herein, refers to the level of the pancreatic cancer or hepatic cancer biomarker in a biological sample such as serum, a gastrointestinal lavage fluid or fecal matter, as determined using a method for the measurement of levels of protein or non-proteinaceous substances. Such methods include, for example, electrophoresis, capillary electrophoresis, high performance liquid chromatography (HPLC), thin layer chromatography (TLC), hyperdiffusion chromatography, fluid or gel precipitation reactions, absorption spectroscopy, a colorimetric assays, spectrophotometric assays, flow cytometry, immunodiffusion (single or double), solution phase assay, immunoelectrophoresis, Western blotting, radioimmunoassay (RIA), enzyme-linked immunosorbent assays (ELISAs), immunofluorescent assays, and electrochemiluminescence immunoassay, and the like. In a preferred embodiment, the level is determined using an ELISA based assay.

The term “sample” as used herein refers to a collection of similar fluids, cells, or tissues isolated from a subject, as well as fluids, cells, or tissues present within a subject. In preferred embodiments, the sample is a biological fluid containing pancreatic cancer or hepatic cancer biomarker. Examples of biological fluids include gastrointestinal lavage fluid, fecal matter, blood, serum and serosal fluids, plasma, semen, pancreatic fluid, bile, lymph, urine, cerebrospinal fluid, saliva, ocular fluids, cystic fluid, tear drops, sputum, mucosal secretions of the secretory tissues and organs, vaginal secretions, gynecological fluids, ascites fluids such as those associated with non-solid tumors, fluids of the pleural, pericardial, peritoneal, abdominal and other body cavities, fluids collected by bronchial lavage and the like. In a particular embodiment, the biological sample is serum, gastrointestinal lavage fluid or fecal matter.

As used herein, the term “progression of a pancreatic disease” or “progression of a hepatic disease” refers to the gradual worsening of the disease over time, whereby symptoms and neurochemical deficits become increasingly more debilitating and/or intense.

As used herein, the term “inhibiting progression of a pancreatic disease” or “inhibiting progression of a hepatic disease” refers to slowing and/or stopping the progression of symptoms of a pancreatic disease or hepatic disease, respectively.

As used herein, “delaying development” of a pancreatic disease, such as pancreatic steatosis, or pancreatic cancer, or of a hepatic disease, such as non-alcoholic fatty liver (NAFL), non-alcoholic steatohepatitis (NASH), or hepatic cancer, means to defer, hinder, slow, retard, stabilize, and/or postpone development of one or more respective symptoms, of the disease, including decreasing the rate at which the patient's disease progresses (e.g., to shift the patient from rapidly progressing disease to a more slowly progressing disease). This delay can be of varying lengths of time, depending on the history of the disorder and/or the medical profile of the individual being treated. As is evident to one skilled in the art, a sufficient or significant delay can, in effect, encompass prevention, in that the individual does not develop detectable disease. A method that “delays” development of disease is a method that reduces the extent of the disease in a given time frame, when compared to not using the method. Such comparisons are typically based on clinical studies, using a statistically significant number of subjects, although this knowledge can be based upon anecdotal evidence. “Delaying development” can mean that the extent and/or undesirable clinical manifestations are lessened and/or time course of the progression is slowed or lengthened, as compared to not administering the agent. Thus, the term also includes, but is not limited to, alleviation of symptoms, diminishment of extent of disease, stabilized (i.e., not worsening) state of disease, delay or slowing of disease progression, and remission (whether partial or total) whether detectable or undetectable.

As used herein an “effective amount” refers to a dosage of an agent sufficient to provide a medically desirable prophylactic and/or therapeutic effect on a pancreatic disease (e.g., pancreatic steatosis and a pancreatic cancer such as pancreatic duct adrenal carcinoma (PDAC)) or a hepatic disease (e.g., NAFL, NASH, and a hepatic cancer such as Hepatocellular carcinoma (HCC)). The effective amount will vary with the desired outcome, the particular pancreatic disease or hepatic disease being treated (or prevented), the age and physical condition of the subject being treated, the severity of the condition, the duration of the treatment, the nature of the concurrent or combination therapy (if any), the specific route of administration and like factors within the knowledge and expertise of the health practitioner. An “effective amount” can be determined empirically and in a routine manner, in relation to the stated purpose. A prophylactic and/or therapeutic effect relating to a cancer includes, but is not limited to, a decrease in tumor size, a suppression or decrease in tumor growth, no new tumor formation, a decrease in new tumor formation, an increase in survival or progression-free survival, no metastases, an increase in treatment options, a decrease in biomarkers typically elevated in the cancer, and a delay in time from surgery to recurrence.

The terms “subject”, “patient,” “individual,” and “animal” are used interchangeably and refer to mammals such as human patients and non-human primates, as well as experimental animals such as rabbits, rats, and mice, and other laboratory animals. Animals include all vertebrates, e.g., mammals and non-mammals, such as chickens, amphibians, and reptiles. “Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and other members of the class Mammalia known in the art. In a particular embodiment, the patient is a human.

Terms such as “treating.” or “treatment,” “to treat,” or “therapy.” refer to both (a) therapeutic measures that cure, slow down, attenuate, lessen symptoms of, and/or halt progression of a pathologic condition and (b) prophylactic or preventative measures that prevent and/or slow the development of a targeted condition and or its related symptoms.

Thus, subjects in need of treatment include those already with the pancreatic disease and/or hepatic disease; those at risk of having the pancreatic disease and/or hepatic disease; and those in whom the pancreatic disease and/or hepatic disease is to be prevented. Subjects can routinely be identified as “having or at risk of having” a pancreatic disease and/or hepatic disease or another condition referred to herein using medical and diagnostic techniques known in the art. In certain embodiments, a subject is successfully “treated” according to the provided methods if the subject shows, e.g., total, partial, or transient amelioration or elimination of at least one symptom associated with the condition.

In other embodiments, the terms “treating,” or “treatment,” “to treat,” or “therapy,” refer to the inhibition of the progression of an pancreatic disease and/or hepatic disease, either physically by, e.g., stabilization of a discernible symptom, physiologically by, e.g., stabilization of a physical parameter, or both. In other embodiments, the terms “treating,” or “treatment,” “to treat,” or “therapy,” refer to the reduce the alleviation of symptoms, the reduction of inflammation, the restoration of cell function. Treatment can be with the HIF1-α Pathway Inhibitor and PFKFB3 inhibitor compositions disclosed herein, or in further combination with one or more additional therapeutic agent.

The term “pharmaceutically acceptable carrier” refers to an ingredient in a pharmaceutical formulation, other than an active ingredient, which is nontoxic to a subject. A pharmaceutically acceptable carrier includes, but is not limited to, a buffer, carrier, excipient, stabilizer, diluent, or preservative. Pharmaceutically acceptable carriers can include for example, one or more compatible solid or liquid filler, diluents or encapsulating substances which are suitable for administration to a human or other subject.

“Therapeutic agent(s)” used according to the disclosed methods and compositions can additionally include any agent directed to treat a condition in a subject. Therapeutic agents” also refer to salts, acids, and free based forms of the above agents.

### PFKFB3 Inhibitors

PFKFB3 (6-phosphofructo-2-kinase-fructose-2,6-bisphosphatase 3) is a bifunctional protein that is involved in both the synthesis and degradation of fructose-2,6-bisphosphate, a regulatory molecule that controls glycolysis in eukaryotes and is required for cell cycle progression and the prevention of apoptosis.

In some embodiments, the disclosure provides a method of treating a pancreatic disease or hepatic disease in a subject in need thereof that comprises:
(a) administering an effective amount of a HIF1-α Pathway Inhibitor or a HIF1-α Inhibitor and an PFKFB3 inhibitor to the subject;
(b) administering an effective amount of a HIF1-α Pathway Inhibitor or a HIF1-α Inhibitor to the subject, wherein the subject has previously been administered a PFKFB3 Inhibitor; or
(c) administering an effective amount of a PFKFB3 Inhibitor to the subject wherein the subject has previously been administered a HIF1-α Pathway Inhibitor or a HIF1-α Inhibitor to the subject;
wherein the PFKFB3 Inhibitor does not inhibit the PI3K/AKT/mTOR pathway or HIF1-α.

The PFKFB3 Inhibitors that can be used according to the provided methods are not particularly limited. In some embodiments, the administered PFKFB3 Inhibitor is an antibody or a PFKFB3-binding antibody (e.g., a single chain antibody, a single-domain antibody, a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, miRNA, dsRNA, ssRNA, and shRNA), a peptibody, a nanobody, a PFKFB3 inhibitory binding polypeptide, or a small molecule PFKFB3 Inhibitor.

In some embodiments, the PFKFB3 inhibitor administered according to the provide methods has an IC50 for a PFKFB3 activity/function of 100 μM or lower concentration for a PFKFB3 activity. In some embodiments, the PFKFB3 inhibitor has an IC50 of at least or at most or about 200, 100, 80, 50, 40, 20, 10, 5, or 1 μM, or at least or at most or about 100, 10, or 1 nM, or lower (or any range or value derivable therefrom). In some embodiments, the PFKFB3 inhibitor inhibits the expression of PFKFB3. Assays for determining the ability of a compound to inhibit PFKFB3 activity are known in the art. In some embodiments, the inhibition of PFKFB3 activity or expression is a decrease as compared with a control level or sample. In some embodiments, a functional assay such as an MTT assay, cell proliferation assay, BRDU or Ki67 immunofluorescence assay, apoptosis assay, or glycolysis assay is used to assay for the ability of a composition to inhibit PFKFB3 activity.

In some embodiments, the PFKFB3 Inhibitor administered according to the provided methods is an antibody or a PFKFB3-binding antibody fragment (e.g., a single chain antibody, a single-domain antibody, a Fab fragment, F(ab′)2 fragment, Fd fragment; Fv fragment, scFv, dAb fragment, or another engineered molecule, such as a diabody, triabody, tetrabody, minibody, and a minimal recognition unit), In particular embodiments, the administered PFKFB3 Inhibitor is a nanobody (e.g., a VHH).

In some embodiments, the HIF1-A Inhibitor administered according to the provided methods is a therapeutic nucleic acid. In some embodiments the therapeutic nucleic acid is an aptamer, antisense molecule, ribozyme, a Dicer substrate, miRNA, dsRNA, ssRNA, and shRNA). In particular embodiments, the HIF1-alpha Inhibitor administered according to the provided methods is an siRNA or an antisense oligonucleotide. In one embodiment, the administered PFKFB3 Inhibitor is EZN-4178.

Representative examples of human PFKFB3 coding sequences are provided in GenBank accession numbers NM_004566.3, NM_001145443.2, NP_001138915.1, NM_001282630.2, NM_001314063.1, NM_001323016.1, NM_001323017.1, and NM_001363545.2. The sequences associated with the each of these Genbank accession numbers is hereby incorporated by reference herein in its entirety for all purposes. Therapeutic nucleic acids that inhibit PFKFB3 activity can routinely be designed and prepared based on each of the above human PFKFB3 transcript sequences using methods known in the art.

The administration of PFKFB3 inhibitory nucleic acids or any ways of inhibiting gene expression of PFKFB3 known in the art are contemplated in certain embodiments of the provided methods. Examples of inhibitory nucleic acid include but are not limited to, antisense nucleic acids such as: small interfering RNA (SiRNA), short hairpin RNA (shRNA), double-stranded RNA, and any other antisense oligonucleotide. Also included are ribozymes or nucleic acids encoding any of the inhibitors described herein. An inhibitory nucleic acid may inhibit the transcription of PFKFB3 or prevent the translation of a PFKFB3 gene transcript in a cell. In some embodiments, the PFKFB3 inhibitory nucleic acid administered according to the provided methods is from 16 to 1000 nucleotides in length. In certain embodiments the administered PFKFB3 inhibitory nucleic acid is from 18 to 100 nucleotides long. In certain embodiments the administered PFKFB3 inhibitory nucleic acid at least or at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 50, 60, 70, 80, 90 nucleotides or any range derivable therefrom.

In some embodiments, the PFKFB3 inhibitory nucleic acid administered according to the provided methods is capable of decreasing the expression of PFKFB3 by at least 10%, 20%, 30%, or 40%, more particularly by at least 50%, 60%, or 70%, and most particularly by at least 75%, 80%, 90%, 95% or more or any range or value in between the foregoing.

In some embodiments, the PFKFB3 inhibitory nucleic acid administered according to the provided methods is between 17 to 25 nucleotides in length and comprises a 5′ to 3′ sequence that is at least 90% complementary to the 5′ to 3′ sequence of a mature PFKFB3 mRNA (e.g., a sequence as disclosed in any one or more of GenBank accession nos. NM_004566.3, NM_001145443.2, NM_001282630.2, NM_001314063.1, NM_001323016.1, NM_001323017.1, and NM_001363545.2). In some embodiments, the administered PFKFB3 inhibitory nucleic acid is 17, 18, 19, 20, 21, 22, 23, 24, or 25 nucleotides in length, or any range derivable therein. In some embodiments, the administered PFKFB3 inhibitory nucleic acid has a sequence (from 5′ to 3′) that is at least 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the corresponding 5′ to 3′ sequence of a mature PFKFB3 mRNA (e.g., a sequence as disclosed in any one or more of GenBank accession nos. NM_004566.3, NM_001145443.2, NM_001282630.2, NM_001314063.1, NM_001323016.1, NM_001323017.1, and NM_001363545.2). One of skill in the art could use a portion of the probe sequence that is complementary to the sequence of a mature mRNA as the sequence for an mRNA inhibitor. Moreover, that portion of the probe sequence can be altered so that it is still 90% complementary to the sequence of a mature mRNA.

In some embodiments, the PFKFB3 inhibitory nucleic acid administered according to the provided methods is a miRNA. In further embodiments, the administered miRNA is a member selected from: hsa-mir-26b-5p (MIRT028775), hsa-mir-330-3p (MIRT043840), hsa-mir-6779-5p (MIRT454747), hsa-mir-6780a-5p (MIRT454748), hsa-mir-3689c (MIRT454749), hsa-mir-3689b-3p (MIRT454750), hsa-mir-3689a-3p (MIRT454751), hsa-mir-30b-3p (MIRT454752), hsa-mir-1273h-5p (MIRT454753), hsa-mir-6778-5p (MIRT454754), hsa-mir-1233-5p (MIRT454755), hsa-mir-6799-5p (MIRT454756), hsa-mir-7106-5p (MIRT454757), hsa-mir-6775-3p (MIRT454758), hsa-mir-1291 (MIRT454759), hsa-mir-765 (MIRT454760), hsa-mir-423-5p (MIRT454761), hsa-mir-3184-5p (MIRT454762), hsa-mir-6856-5p (MIRT454763), hsa-mir-6758-5p (MIRT454764), hsa-mir-3185 (MIRT527973), hsa-mir-6892-3p (MIRT527974), hsa-mir-6840-5p (MIRT527975), and hsa-mir-6865-3p (MIRT527976).

In some embodiments, the PFKFB3 inhibitor administered according to the provide methods is a small molecule. The administered small molecule PFKFB3 inhibitors may be any small molecules that is determined to inhibit PFKFB3 function or activity. Such small molecules may be determined based on functional assays in vitro or in vivo.

In some embodiments, the PFKFB3 inhibitor small molecules administered according to the provide methods is a small molecule PFKFB3 inhibitory molecules disclosed in U.S. publication nos. 20130059879, 20120177749, 20100267815, 20100267815, and 20090074884, the disclosure of each of which is herein incorporated by reference in its entirety.

In some embodiments, the PFKFB3 inhibitor administered according to the provided methods is at least one of: (1H-Benzo[g]indol-2-yl)-phenyl-methanone; (3H-Benzo[e]indol-2-yl)-phenyl-methanone; (3H-Benzo[e]indol-2-yl)-(4-methoxy-phenyl)-methanone; (3H-Benzo[e]indol-2-yl)-pyridin-4-yl-methanone; HCl salt of (3H-Benzo[e]indol-2-yl)-pyridin-4-yl-methanone; (3H-Benzo[e]indol-2-yl)-(3-methoxy-phenyl)-methanone; (3H-Benzo[e]indol-2-yl)-pyridin-3-yl-methanone; (3H-Benzo[e]indol-2-yl)-(2-methoxy-phenyl)-methanone; (3H-Benzo[e]indol-2-yl)-(2-hydroxy-phenyl)-methanone; (3H-Benzo[e]indol-2-yl)-(4-hydroxy-phenyl)-methanone; (5-Methyl-3H-benzo[e]indol-2-yl)-phenyl-methanone; Phenyl-(7H-pyrrolo[2,3-h]quinolin-8-yl)-methanone; (3H-Benzo[e]-indol-2-yl)-(3-hydroxy-phenyl)-methanone; (3H-benzo[e]indol-2-yl)-(2-chloro-pyridin-4-yl)-methanone; (3H-benzo[e]indol-2-yl)-(1-oxy-pyridin-4-yl)-methanone; Phenyl-(6,7,8,9-tetrahydro-3H-benzo[e]indol-2-yl)-methanone; (3H-Benzo[e]indol-2-yl)-(4-hydroxy-3-methoxylthenyl)-methanone; (3H-Benzo[e]indol-2-yl)-(4-benzyloxy-3-methoxy-phen-yl)-methanone; 4-(3H-Benzo[e]indole-2-carbonyl)-benzoic acid methyl ester; 4-(3H-Benzo[e]indole-2-carbonyl)-benzoic acid; (4-Amino-phenyl)-(3H-benzo[e]indol-2-yl)-methanone; 5-(3H-Benzo[e]indole-2-carbonyl)-2-benzyloxy-benzoic acid methyl; 5-(3H-Benzo[e]indole-2-carbonyl)-2-benzyloxy-benzoic Acid methanone; (3H-Benzo[e]indol-2-yl)-(2-methoxy-pyridin-4-yl)-methanone; (5-Fluoro-3H-benzo[e]indol-2-yl)-(3-methoxy-phenyl)-methanone; (5-Fluoro-3H-benzo[e]indol-2-yl)-pyridin-4-yl-methanone; (4-Benz-yloxy-3-methoxy-phenyl)-(5-fluoro-3H-benzo[e]indol-2-yl)-methanone; (5-Fluoro-3H-benzo[e]indol-2-yl)-(4-hydroxy-3-methoxy-phenyl)-methanone; (3H-Benzo[e]indol-2-yl)-(3-hydroxymethyl-phenyl)-methanone; Cyclohexyl-(5-fluoro-3H-benzo[e]indol-2-yl)-methanone; (5-Fluoro-3H-benzo[e]indol-2-yl)-(3-fluoro-4-hydroxy-phenyl)-methanone; (3H-Benzo[e]indol-2-yl)-p-tolyl-methanone; (3H-Benzo[e]indol-2-yl)-(3-methoxy-phen-yl-methanol; (3H-Benzo[e]indol-2-yl)-pyridin-4-yl-methanol; 3H-Benzo[e]indole-2-carboxylic acid phenylamide; 3H-Benzo[e]indole-2-carboxylic acid (3-methoxy-phenyl)-amide; (3H-Benzo[e]indol-2-yl)-(4-dimethylamino-phenyl)-methanone; (4-Amino-3-meth-oxy-phenyl)-(3H-benzo[e]indol-2-yl)-methanone; (4-Amino-3-methoxy-phenyl)-(5-hydroxy-3H-benzo[e]indol-2-yl)-methanone; (4-Amino-3-methoxy-phenyl)-(5-methoxy-3H-benzo[e]indol-2-yl)-methanone; N-[4-(3H-Benzo[e]indole-2-carbonyl)-phenyl]-methanesulfonamide; 3H-Benzo[e]indole-2-carboxylic acid (4-amino-phenyl)-amide; (4-Amino-phenyl)-(5-methoxy-3H-benzo[e]indol-2-yl)-methanone; (4-Amino-2-fluoro-phenyl)-(5-methoxy-3H-benzo[e]indol-2-yl)-methanone; (4-Amino-3-fluoro-phenyl)-(5-methoxy-3H-benzo[e]indol-2-yl)-methanone; (4-Amino-2-methoxy-phenyl)-(5-methoxy-3H-benzo[e]indol-2-yl)-methanone; (4-Amino-phenyl)-(9-methoxy-3H-benzo[e]indol-2-yl)-methanone; (4-Amino-3-methoxy-phenyl)-(9-methoxy-3H-benzo[e]indol-2-yl)-methanone; (4-Amino-2-methoxy-phenyl)-(9-methoxy-3H-benzo[e]indol-2-yl)-methanone; (4-Amino-3-fluoro-phenyl)-(9-methoxy-3H-benzo[e]indol-2-yl)-methanone; (4-Amino-2-fluoro-phenyl)-(9-methoxy-3H-benzo[e]indol-2-yl)-methanone; (4-Amino-3-fluoro-phen-yl)-(3H-benzo[e]indol-2-yl)-methanone; (4-Amino-2-fluoro-phenyl)-(3H-benzo[e]indol-2-yl)-methanone; (4-Amino-phenyl)-(7-methoxy-3H-benzo[e]indol-2-yl)-methanone; (4-Amino-phenyl)-(5-hydroxy-3-methyl-3H-benzo[e]indol-2-yl)-methanone; (7-Amino-5-fluoro-9-hydroxy-3H-benzo[e]indol-2-yl)-(3-methyl-pyridin-4-yl)-methanone; (5-Amino-3H-pyrrolo[3,2-f]isoquinolin-2-yl)-(3-methoxy-pyridin-4-yl)-methanone; (4-Amino-2-methyl-phenyl)-(9-hydroxy-3H-pyrrolo[2,3-c]quinolin-2-yl)-methanone; and (4-Amino-phenyl)-(7-methanesulfonyl-3H-benzo[e]indol-2-yl)-methanone, or a salt thereof.

In some embodiments, the PFKFB3 inhibitor administered according to the provided methods is at least one of: 1-Pyridin-4-yl-3-quinolin-4-yl-propenone; 1-Pyridin-4-yl-3-quinolin-3-yl-propenone; 1-Pyridin-3-yl-3-quinolin-2-yl-propenone; 1-Pyridin-3-yl-3-quinolin-4-yl-propenone; l-Pyridin-3-yl-3-quinolin-3-yl-propenone; l-Naphthalen-2-yl-3-quinolin-2-yl-propenone; l-Naphthalen-2-yl-3-quinolin-3-yl-propenone; l-Pyridin-4-yl-3-quinolin-3-yl-propenone; 3-(4-Hydroxy-quinolin-2-yl)-1-pyridin-4-yl-propenone; 3-(8-Hydroxy-quinolin-2-yl)-1-pyridin-3-yl-propenone; 3-Quinolin-2-yl-1-p-tolyl-propenone; 3-(8-Hydroxy-quinolin-2-yl)-1-pyridin-4-yl-propenone; 3-(8-Hydroxy-quinolin-2-yl)-1-p-tolyl-propenone; 3-(4-Hydroxy-quinolin-2-yl)-1-p-tolyl-propenone; 1-Phenyl-3-quinolin-2-yl-propenone; 1-Pyridin-2-yl-3-quinolin-2-yl-propenone; 1-(2-Hydroxy-phenyl)-3-quinolin-2-yl-propenone; 1-(4-Hydroxy-phenyl)-3-quinolin-2-yl-propenone; 1-(2-Amino-phenyl)-3-quinolin-2-yl-propenone; 1-(4-Amino-phenyl)-3-quinolin-2-yl-propenone; or a salt thereof.

In some embodiments, the PFKFB3 inhibitor administered according to the provided methods is at least one of: 4-(3-Quinolin-2-yl-acryloyl)-benzamide; 4-(3-Quinolin-2-yl-acryloyl)-benzoic acid; 3-(8-Methyl-quinolin-2-yl)-1-pyridin-4-yl-propenone; 1-(2-Fluoro-pyridin-4-yl)-3-quinolin-2-yl-propenone; 3-(8-Fluoro-quinolin-2-yl)-1-pyridin-4-yl-propenone; 3-(6-Hydroxy-quinolin-2-yl)-1-pyridin-4-yl-propenone; 3-(8-Methylamino-quinolin-2-yl)-1-pyridin-4-yl-propenone; 3-(7-Methyl-quinolin-2-yl)-1-pyridin-4-yl-propenone; and 1-Methyl-4-[3-(8-methyl-quinolin-2-yl)-acryloyl]-pyridinium, or a salt thereof.

In some embodiments, the PFKFB3 inhibitor administered according to the provided methods is at least one of: PFK15 (1-(4-pyridinyl)-3-(2-quinolinyl)-2-propen-1-one); (2S)—N-[4-[[3-Cyano-1-(2-methyl-propyl)-1H-indol-5-yl]oxy]phenyl]-2-pyrrolidine-carboxamide 3PO (3-(3-Pyridinyl)-1-(4-pyridinyl)-2-propen-1-one); (2S)—N-[4-[[3-Cyano-1-[(3,5-dimethyl-4-isoxazolyl)methyl]-1H-indol-5-yl]oxy]phenyl]-2-pyrrolidine-carboxamide; and Ethyl 7-hydroxy-2-oxo-2H-1-benzopyran-3-carboxylate, or a salt thereof.

In a particular embodiment, the PFKFB3 inhibitor administered according to the provided methods is PFK15, or a salt thereof.

In a particular embodiment, the PFKFB3 inhibitor administered according to the provided methods is PFK158 ((E)-1-(4-Pyridinyl)-3-[7-(trifluoromethyl)-2-quinolinyl]-2-propen-1-one), or a salt thereof.

In a particular embodiment, the PFKFB3 inhibitor administered according to the provided methods is BrAcNHEtOP (N-bromoacetylethanolamine phosphate), or a salt thereof.

In a particular embodiment, the PFKFB3 inhibitor administered according to the provided methods is AZ67, or a salt thereof.

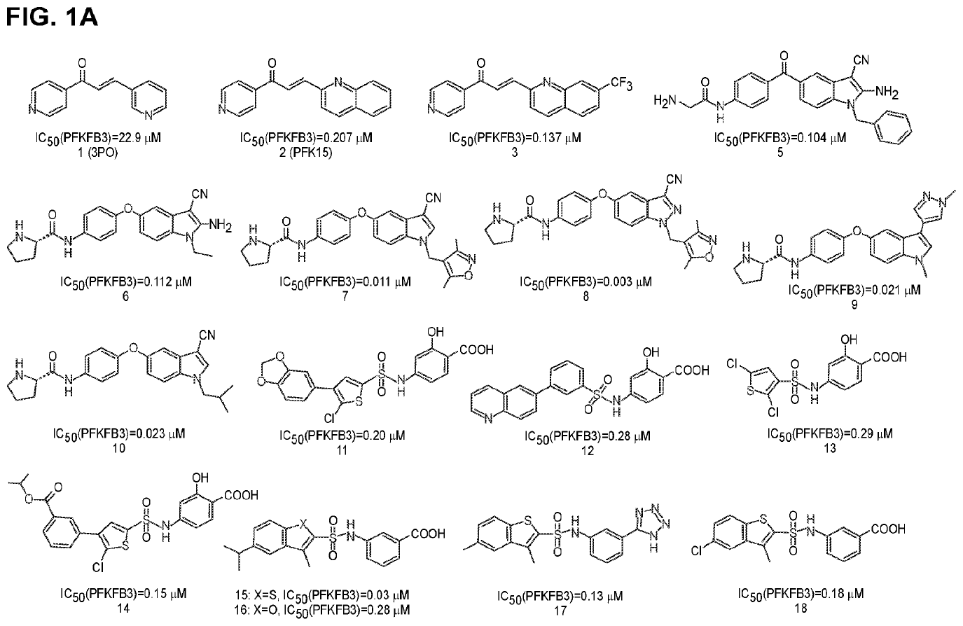

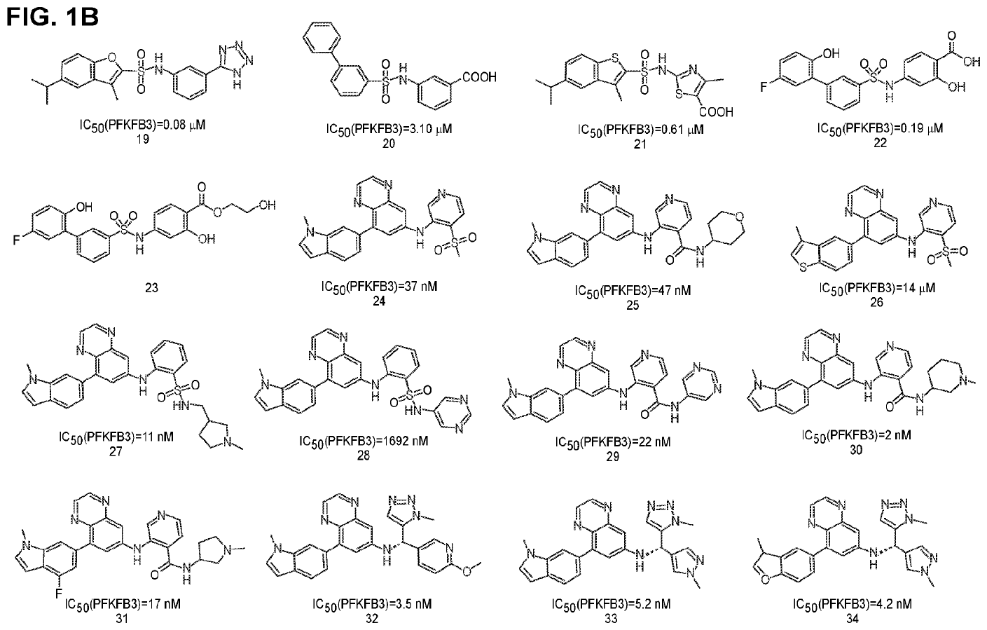

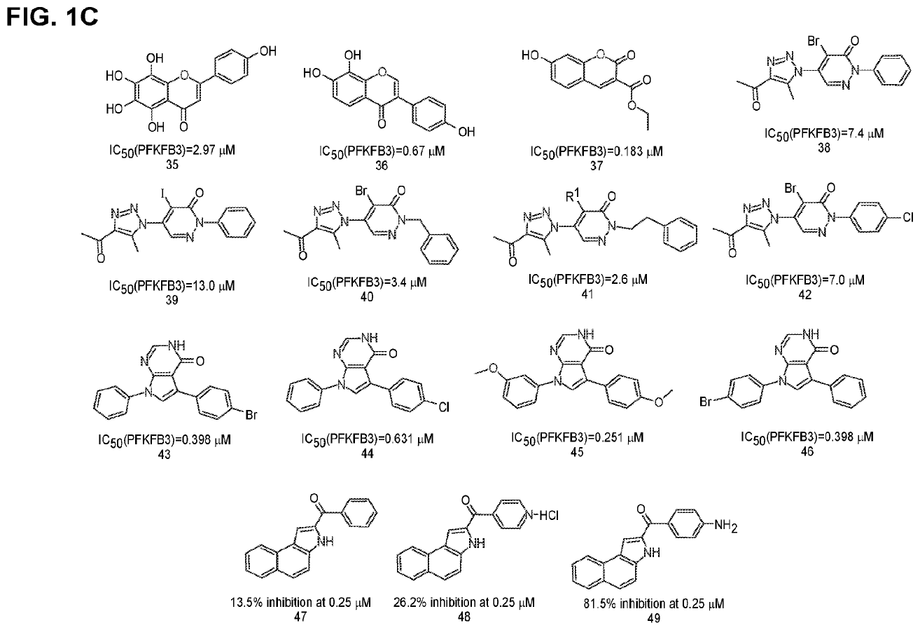

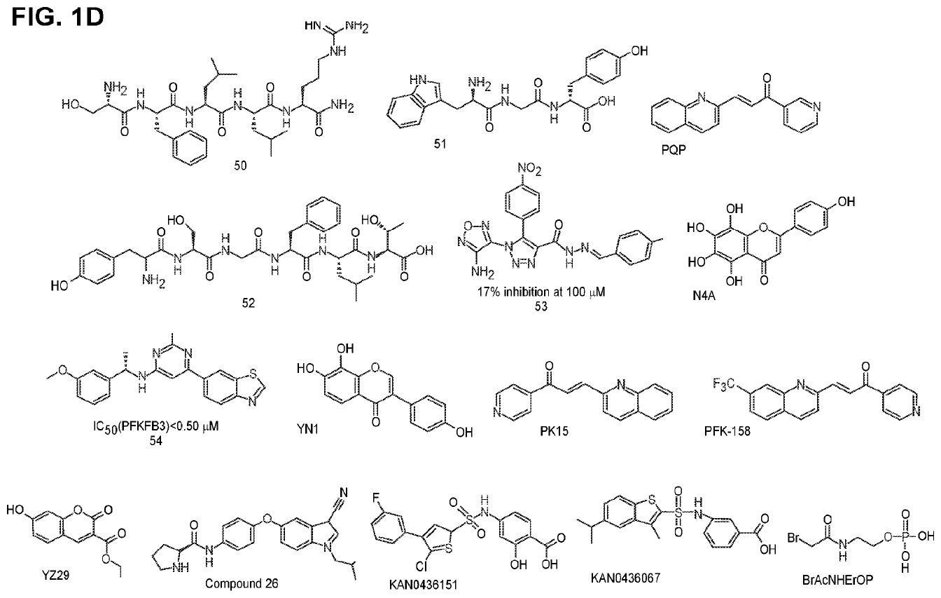

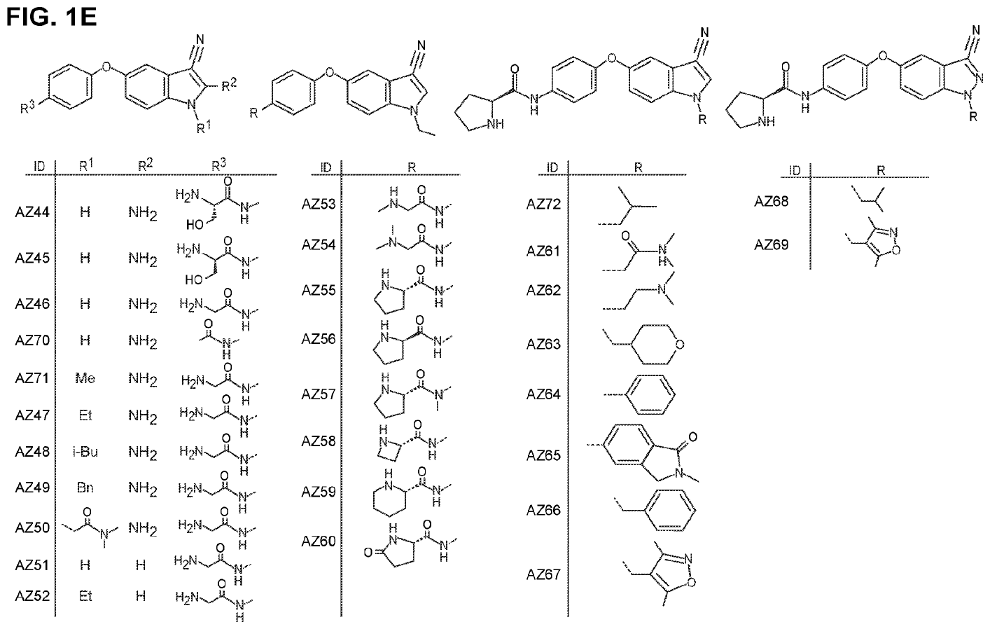

In some embodiments, the PFKFB3 inhibitor administered according to the provided methods is at least one PFKFB3 inhibitor having the structure of formula 1-53 or 54, PQP, N4A, YN1, PK15, PFK-158, YZ29, Compound 26, KAN0436151, KAN0436067, or BrAcNHErOP, depicted in FIG. 1A-1D, or a salt thereof. In some embodiments, the PFKFB3 inhibitor administered according to the provided methods is the PFKFB3 inhibitor having the structure of formula AZ44-AZ70 or AZ71, depicted in FIG. 1E, or a salt thereof.

In a particular embodiment, the PFKFB3 inhibitor administered according to the provided methods is KAN0436151, or a salt thereof.

In a particular embodiment, the PFKFB3 inhibitor administered according to the provided methods is KAN0436067, or a salt thereof.

### HIF1-α Pathway Inhibitors

Hypoxia-inducible factor 1-alpha (HIF-1-alpha) is a subunit of a heterodimeric transcription factor hypoxia-inducible factor 1 (HIF-1) that is considered to be the master transcriptional regulator of cellular and developmental response to hypoxia.

In some embodiments, the disclosure provides a method of treating a pancreatic disease and/or hepatic disease in a subject in need thereof that comprises:
(a) administering an effective amount of a HIF1-α Pathway Inhibitor or a HIF1-α Inhibitor and an PFKFB3 inhibitor to the subject;
(b) administering an effective amount of a HIF1-α Pathway Inhibitor or a HIF1-α Inhibitor to the subject, wherein the subject has previously been administered a PFKFB3 Inhibitor; or
(c) administering an effective amount of a PFKFB3 Inhibitor to the subject wherein the subject has previously been administered a HIF1-α Pathway Inhibitor or a HIF1-α Inhibitor; and
wherein the PFKFB3 inhibitor does not inhibit PI3K/AKT/mTOR pathway or HIF1-α.

The term “HIF1-α Pathway-α Inhibitor” as used herein refers to a composition that inhibits or reduces HIF1-α directly or indirectly via inhibiting one or more activities of the PI3K/AKT/mTOR pathway that is upstream of the HIF1-α pathway. The term “HIF1-α Inhibitor” is used herein to refer to a composition that inhibits or reduces HIF1-α directly. Thus, for example, mTOR pathway inhibitors such as temsirolimus, everolimus, and sirolimus are considered herein to be “HIF1-α Pathway-α Inhibitors”, but not “HIF1-α Inhibitors.”

The “HIF1-α Pathway-α Inhibitors” that can be administered according to the provided methods are not particularly limited. In some embodiments, the administered HIF1-α Pathway Inhibitor is an antibody or a HIF1-α-binding antibody fragment (e.g., a single chain antibody, a single-domain antibody (e.g., a VHH), a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, ENMD-1198, miRNA, dsRNA, ssRNA, and shRNA), a peptibody, a nanobody, a HIF1-α Pathway binding polypeptide, or a small molecule HIF1-α Pathway Inhibitor.

In some embodiments, the administered HIF1-α Pathway Inhibitor administered according to the provided methods has an IC50 for a HIF1-α activity/function of 100 M or lower concentration for a HIF1-α activity. In some embodiments, the HIF1-α Pathway Inhibitor has an IC50 of at least or at most or about 200, 100, 80, 50, 40, 20, 10, 5, or 1 M, or at least or at most or about 100, 10, or 1 nM, or lower (or any range or value derivable therefrom). In some embodiments, the HIF1-α Pathway Inhibitor inhibits the expression of HIF1-α. Assays for determining the ability of a compound to inhibit HIF1-α activity are known in the art. In some embodiments, the inhibition of HIF1-α activity or expression is a decrease as compared with a control level or sample. In some embodiments, a functional assay such as an MTT assay, cell proliferation assay, BRDU or Ki67 immunofluorescence assay, apoptosis assay, or glycolysis assay is used to assay for the ability of a composition to inhibit HIF1-α activity.

The HIF1-α Inhibitors that can be administered according to the provided methods are not particularly limited. In some embodiments, the HIF1-α Inhibitor modulates one or more of HIF-1α mRNA expression; HIF-1α protein translation or degradation; HIF-1α/HIF-1β dimerization; HIF-1α-DNA binding (e.g., HIF-1α/HRE); and/or HIF-1α transcriptional activity (e.g., CH-1 of p300/C-TAD of HIF-1α).

In some embodiments, the HIF1-α Inhibitor administered according to the provided methods is a small molecule. In some embodiments, the HIF1-α Inhibitor administered according to the provided methods is a protein or polypeptide (e.g., an anti HIF1 antibody or antibody fragment that binds HIF1). In some embodiments, the HIF1-α Inhibitor administered according to the provided methods is a therapeutic nucleic acid (e.g., an aptamer, antisense molecule, ribozyme, a Dicer substrate, siRNA, miRNA, dsRNA, ssRNA, or an shRNA).

In some embodiments, the HIF1-α Pathway Inhibitor administered according to the provided methods is a HIF1-α Pathway Inhibitor (e.g., a PI3K pathway inhibitor, a MAPK pathway inhibitor, an Akt pathway inhibitor, and/or an mTOR inhibitor); a HIF translation inhibitor (e.g., a topoisomerase inhibitor, a microtubule targeting drug a cardiac glycoside, or an antisense HIF-1α mRNA); an inhibitor of HIF stability, nuclear localization or dimerization (e.g., acriflavine or an HDAC inhibitor); an inhibitor of HIF transactivation (e.g., a HIF1 coactivator recruitment inhibitor or a HIF1 DNA binding inhibitor).

In some embodiments, the HIF1-α Inhibitor administered according to the provided methods is a HIF1-α Pathway Inhibitor (e.g., a PI3K pathway inhibitor, a MAPK pathway inhibitor, an Akt pathway inhibitor, and/or an mTOR inhibitor). In some embodiments, the HIF1-α Inhibitor administered according to the provided methods is a PI3K pathway inhibitor. In one embodiment, the administered HIF1-α Pathway Inhibitor is P3155, LY29, LY294002, wortmannin, or GDC-0941. In one embodiment, the administered HIF1-α Pathway Inhibitor is resveratrol. In another embodiment, the administered HIF1-α Pathway Inhibitor is a glyceolin. In some embodiments, the HIF1-α Pathway Inhibitor administered according to the provided methods is an mTOR inhibitor. In one embodiment, the administered HIF1-α Pathway Inhibitor is rapamycin, temsirolimus (CC1-779), everolimus, sirolimus, or PP242.

In a particular embodiment, the administered HIF1-α Inhibitor is silibinin.

In some embodiments, the HIF1-α Inhibitor administered according to the provided methods is a HIF translation inhibitor. In one embodiment, the administered HIF1-α Inhibitor is PX-478 (S-2-amino-3-[4′-N,N-bis(chloroethyl)[amino]phenyl propionic acid N-oxide dihydrochloride), NSC-64421, camptothecin (CPT), SN38, irinotecan, topotecan, NSC-644221, cycloheximide, or apigenin, or a salt thereof. In one embodiment, the administered HIF1-α Inhibitor is aminoflavone, KC7F2 (N,N′-(disulfanediylbis(ethane-2,1-diyl))bis(2,5-dichlorobenzene-sulfonamide), 2-meth-oxyestra-diol (2ME2) or an analog or salt thereof. In one embodiment, the administered HIF1-α Inhibitor is ENMD-1198, ENMD-1200, or ENMD-1237, or a salt thereof. In one embodiment, the administered HIF1-α Inhibitor is EZN-2208, or a salt thereof. In one embodiment, the administered HIF1-α Inhibitor is EZN-2968, or a salt thereof.

In a particular embodiment, the administered HIF1-α Inhibitor is PX-478, or a salt thereof.

In some embodiments, the HIF1-α Inhibitor administered according to the provided methods is a cardiac glycoside. In one embodiment, the administered cardiac glycoside is digoxin, or a salt thereof. In another embodiment, the administered cardiac glycoside ouabain or proscillardin A, or a salt thereof.

In some embodiments, the HIF1-α Pathway Inhibitor administered according to the provided methods is a topoisomerase inhibitor. In one embodiment, the administered topoisomerase inhibitor is camptothecin (CPT), SN38, irinotecan, or topotecan (e.g., PEG-SN38), or a salt thereof.

In some embodiments, the HIF1-α Pathway Inhibitor administered according to the provided methods is a microtubule targeting drug. In one embodiment, the administered microtubule targeting drug is 2 methoxyestradiol (2ME2), ENMD-1198, ENMD-1200, ENMD-1237, or Taxotere, or a salt thereof.

In some embodiments, the HIF1-α Inhibitor administered according to the provided methods is a therapeutic nucleic acid. In some embodiments therapeutic nucleic acid is an aptamer, antisense molecule, ribozyme, a Dicer substrate, siRNA, miRNA, dsRNA, ssRNA, and shRNA). In some embodiments, therapeutic nucleic acid is an antisense oligonucleotide.

In some embodiments, the HIF1-A Inhibitor administered according to the provided methods is a siRNA or an antisense oligonucleotide. In one embodiment, the administered HIF1-α Inhibitor is EZN-2968. In one embodiment, the administered HIF1-α Inhibitor is RX-0047.

Representative examples of human HIF1-A coding sequences are provided in GenBank accession numbers NM_004566.3, NM_001145443.2, NP_001138915.1, NM_001282630.2, NM_001314063.1, NM_001323016.1, NM_001323017.1, and NM_001363545.2. The sequences associated with the each of these Genbank accession numbers is hereby incorporated by reference herein in its entirety for all purposes. Therapeutic nucleic acids that inhibit HIF1-A activity can routinely be designed and prepared based on each of the above human HIF1-A transcript sequences using methods known in the art.

The administration of HIF1-A inhibitory nucleic acids or any ways of inhibiting gene expression of HIF1-A known in the art are contemplated in certain embodiments of the provided methods. Examples of inhibitory (therapeutic) nucleic acid include but are not limited to, antisense nucleic acids such as: a small interfering RNA (siRNA), short hairpin RNA (shRNA), double-stranded RNA, and any other antisense oligonucleotide. Also included are ribozymes or nucleic acids encoding any of the inhibitors described herein. An inhibitory nucleic acid may inhibit the transcription of HIF1-A or prevent the translation of a HIF1-A gene transcript in a cell. In some embodiments, the HIF1-A inhibitory nucleic acid administered according to the provided methods is from 16 to 1000 nucleotides in length. In certain embodiments the administered HIF1-A inhibitory nucleic acid is from 18 to 100 nucleotides long. In certain embodiments the administered HIF1-A inhibitory nucleic acid at least or at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 50, 60, 70, 80, 90 nucleotides or any range derivable therefrom.

In some embodiments, the HIF1-A inhibitory nucleic acid administered according to the provided methods is capable of decreasing the expression of HIF1-A by at least 10%, 20%, 30%, or 40%, more particularly by at least 50%, 60%, or 70%, and most particularly by at least 75%, 80%, 90%, 95% or more or any range or value in between the foregoing.

In some embodiments, the HIF1-A inhibitory nucleic acid administered according to the provided methods is between 17 to 25 nucleotides in length and comprises a 5′ to 3′ sequence that is at least 90% complementary to the 5′ to 3′ sequence of a mature HIF1-A mRNA (e.g., as disclosed in any one or more of GenBank accession nos. NM_001530.4, NM_181054.3, and NM_001243084.2). In some embodiments, the administered HIF1-A inhibitory nucleic acid is 17, 18, 19, 20, 21, 22, 23, 24, or 25 nucleotides in length, or any range derivable therein. In some embodiments, the administered HIF1-A inhibitory nucleic acid has a sequence (from 5′ to 3′) that is at least 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the corresponding 5′ to 3′ sequence of a mature HIF1-A mRNA (e.g., as disclosed in any one or more of GenBank accession nos. NM_001530.4, NM_181054.3, and NM_001243084.2). One of skill in the art could use a portion of the probe sequence that is complementary to the sequence of a mature mRNA as the sequence for an mRNA inhibitor. Moreover, that portion of the probe sequence can be altered so that it is still 90% complementary to the sequence of a mature mRNA.

In some embodiments, the HIF1-A inhibitory nucleic acid administered according to the provided methods is a miRNA mimic. In some embodiments, the administered HIF1-α Inhibitor is a miR-483 mimic.

In some embodiments, the HIF1-α Inhibitor administered according to the provided methods is an inhibitor of HIF stability, nuclear localization or dimerization. In one embodiment, the inhibitor administered according to the provided methods destabilizes HIF. In one embodiment, the inhibitor administered according to the provided methods is a histone deacetylase inhibitor (HDACI). In a further embodiment, the administered HDACI is LW6/CAY10585, vorinostat, romidepsin (FK228), panobinostat, belinostat, Trichostatin A (TSA), LAQ824, or phenethyl isothiocyanate, or a salt thereof. In one embodiment, the inhibitor administered according to the provided methods is PX-12/pleurotin, HIF-1α inhibitor (CAS No. 934593-90-5), cryptotanshinone, or BAY 87-2243 (1-cyclopropyl-4-[4-[[5-methyl-3-[3-[4-(trifluoromethoxy) phenyl]-1,2,4-oxadiazol-5-yl]-1H-pyrazol-1-yl]methyl]-2-pyridinyl]-piperazine), or a salt thereof. In one embodiment, the inhibitor administered according to the provided methods is IDF-11774, Bisphenol A/Dimethyl bisphenol A, or a salt thereof. Chrysin (5,7-dihydroxy-flavone), or SCH66336, or a salt thereof. In one embodiment, the inhibitor administered according to the provided methods is geldanamycin or analog thereof, 17-AAG (tanespimycin: allylamino-17-demethoxygeldanamycin), 17-DMAG (alvespimycin), 17AG, radiccicol, KF58333, ENMD-1198, ENMD-1237, or ganetasipib, or a salt thereof. In one embodiment, the inhibitor administered according to the provided methods interferes with HIF-dimerization. In one embodiment, the inhibitor administered according to the provided methods is acriflavine, or a salt thereof. In one embodiment, the inhibitor administered according to the provided methods is TC-S7009, PT2385, or TAT-cyclo-CLLFVY, or a salt thereof.

In a particular embodiment, the inhibitor administered according to the provided methods is ganetasipib, or a salt thereof.

In a particular embodiment, the inhibitor administered according to the provided methods is BAY 87-2243.

In some embodiments, the HIF1-α Pathway Inhibitor administered according to the provided methods is a histone deacetylase inhibitor (HDACI). In one embodiment, the administered HDACI is LW6/CAY10585 (methyl 3-(2-(4-(adamantan-1-yl)phenoxy) acetamido)-4-hydroxy-benzoate, vorinostat, romidepsin (FK228), panobinostat, belinostat, Trichostatin A (TSA), LAQ824, or phenethyl isothiocyanate, or a salt thereof.

In some embodiments, the HIF1-α Pathway Inhibitor administered according to the provided methods is a heat shock protein inhibitor. In one embodiment, the administered HIF1-α Pathway Inhibitor is an HSP90 inhibitor. In one embodiment, the administered HSP90 inhibitor is a geldanamycin or analog thereof, 17-AAG (tanespimycin: allylamino-17-demethoxy geldanamycin), 17-DMAG (alvespimycin), 17AG, radiccicol, KF58333, ENMD-1198, ENMD-1237, or ganetasipib, or a salt thereof. In a particular embodiment, the administered heat shock protein inhibitor is ganetasipib, or a salt thereof. In one embodiment, the administered HIF1-α Pathway Inhibitor is an HSP70 inhibitor. In one embodiment, the administered HSP70 inhibitor is triptolide, or a salt thereof.

In some embodiments, the HIF1-α Inhibitor administered according to the provided methods is an inhibitor of HIF transactivation. In one embodiment, the HIF1-α Inhibitor administered according to the provided methods inhibits HIF coactivator recruitment. In one embodiment, the administered HIF1-α Inhibitor is chetomin, YC-1, or KCN-1 (3,4-dimethoxy-N-[(2,2-dimethyl-2H-chromen-6-yl)methyl]-N-phenylbenzenesulfonamide), or a salt thereof. In another particular embodiment, the administered HIF1-α Inhibitor is NSC 607097, or a salt thereof. In one embodiment, the administered HIF1-α Inhibitor is a proteasome inhibitor. In a further embodiment, the administered inhibitor is bortezomib or carfilzomib, or a salt thereof. In one embodiment, the administered HIF1-α Inhibitor is indenopyrazole 21, FM19G11, flavopiridol, Amphotericin B, actinomycin, AJM290, or AW464, or a salt thereof. In one embodiment, the administered HIF1-α Inhibitor is triptolide, or a salt thereof.

In a particular embodiment, the HIF1-α Inhibitor administered according to the provided methods is YC-1, or a salt thereof.

In some embodiments, the HIF1-α Inhibitor administered according to the provided methods is an antibody that binds HIF1-α or a HIF1-α-binding antibody fragment (e.g., a single chain antibody, a single-domain antibody (e.g., the AG1-5 VHH), a Fab fragment, F(ab′)2 fragment, Fd fragment; Fv fragment, scFv, dAb fragment, or another engineered molecule, such as a diabody, triabody, tetrabody, minibody, and a minimal recognition unit). In a particular embodiment, the administered HIF1-α Inhibitor is a VHH or nanobody. In one embodiment, the administered antibody is AGI-5. In one embodiment, the administered antibody is AHPC.

In some embodiments, the HIF1-α Inhibitor administered according to the provided methods is an inhibitor of HIF1 DNA-binding. In one embodiment, the administered HIF1-α Inhibitor is echinomycin (NSC-13502) or Compound DJ12.162. In one embodiment, the administered HIF1-α Inhibitor is an anthracycline. In a further embodiment, the administered inhibitor is doxorubicin or danuorubicin. In one embodiment, the administered HIF1-α Inhibitor is a polyamide. In some embodiments, the HIF1-α Inhibitor is an antibody that binds HIF1-α or is a HIF1-α-binding antibody fragment such as a VHH or nanobody.

In some embodiments, the HIF1-A Inhibitor administered according to the provided methods is a therapeutic nucleic acid. In some embodiments the therapeutic nucleic acid is an aptamer, antisense molecule, ribozyme, a Dicer substrate, ENMD-1198, miRNA, dsRNA, ssRNA, and shRNA). In some embodiments, the therapeutic nucleic acid is ENMD-1198 or an antisense oligonucleotide.

In some embodiments, the HIF1-A Inhibitor administered according to the provided methods is an siRNA or an antisense oligonucleotide. In some embodiments, the administered HIF1-A Inhibitor is RX-0047. In some embodiments, the administered HIF1-A Inhibitor is EZN-2968.

Representative examples of human HIF1-A coding sequences are provided in GenBank accession numbers NM_004566.3, NM_001145443.2, NP_001138915.1, NM_001282630.2, NM_001314063.1, NM_001323016.1, NM_001323017.1, and NM_001363545.2. The sequences associated with the each of these Genbank accession numbers is hereby incorporated by reference herein in its entirety for all purposes. Therapeutic nucleic acids that inhibit HIF1-A activity can routinely be designed and prepared based on each of the above human HIF1-A transcript sequences using methods known in the art.

The administration of HIF1-A inhibitory nucleic acids or any ways of inhibiting gene expression of HIF1-A known in the art are contemplated in certain embodiments of the provided methods. Examples of inhibitory nucleic acid include but are not limited to, antisense nucleic acids such as: a small interfering RNA (siRNA), short hairpin RNA (shRNA), double-stranded RNA, and any other antisense oligonucleotide. Also included are ribozymes or nucleic acids encoding any of the inhibitors described herein. An inhibitory nucleic acid may inhibit the transcription of HIF1-A or prevent the translation of a HIF1-A gene transcript in a cell. In some embodiments, the HIF1-A inhibitory nucleic acid administered according to the provided methods is from 16 to 1000 nucleotides in length. In certain embodiments the administered HIF1-A inhibitory nucleic acid is from 18 to 100 nucleotides long. In certain embodiments the administered HIF1-A inhibitory nucleic acid at least or at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 50, 60, 70, 80, 90 nucleotides or any range derivable therefrom.

In some embodiments, the HIF1-A inhibitory nucleic acid administered according to the provided methods is capable of decreasing the expression of HIF1-A by at least 10%, 20%, 30%, or 40%, more particularly by at least 50%, 60%, or 70%, and most particularly by at least 75%, 80%, 90%, 95% or more or any range or value in between the foregoing.

In some embodiments, the HIF1-A inhibitory nucleic acid administered according to the provided methods is between 17 to 25 nucleotides in length and comprises a 5′ to 3′ sequence that is at least 90% complementary to the 5′ to 3′ sequence of a mature HIF1-A mRNA (e.g., as disclosed in any one or more of GenBank accession nos. NM_001530.4, NM_181054.3, and NM_001243084.2). In some embodiments, the administered HIF1-A inhibitory nucleic acid is 17, 18, 19, 20, 21, 22, 23, 24, or 25 nucleotides in length, or any range derivable therein. In some embodiments, the administered HIF1-A inhibitory nucleic acid has a sequence (from 5′ to 3′) that is at least 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the corresponding 5′ to 3′ sequence of a mature HIF1-A mRNA (e.g., as disclosed in any one or more of GenBank accession nos. NM_001530.4, NM_181054.3, and NM_001243084.2). One of skill in the art could use a portion of the probe sequence that is complementary to the sequence of a mature mRNA as the sequence for an mRNA inhibitor. Moreover, that portion of the probe sequence can be altered so that it is still 90% complementary to the sequence of a mature mRNA.

In some embodiments, the HIF1-A inhibitory nucleic acid administered according to the provided methods is a miRNA mimic. In some embodiments, the administered HIF1-α Inhibitor is a miR-483 mimic.

In some embodiments, the HIF1-α Inhibitor administered according to the provided methods is a therapeutic nucleic acid. In some embodiments the therapeutic nucleic acid is an ENMD-1198 molecule or antisense oligonucleotide.

### GPR81 Inhibitors

Lactate receptor (a.k.a. G protein-coupled receptor 81, GPR81, Hydroxy-carboxylic acid receptor 1 and HCA1) is a G protein-coupled receptor that has been reported to play a central role in promoting aberrant tissue remodeling as well as cancer induced angiogenesis, immune evasion, and chemoresistance. In the context of the present disclosure, the chronic HIFla-PFKFB3-dependent release of lactate from aberrant cells in the pancreas plays an autocrine role in promoting aberrant function of cells in the pancreas and in propagating pancreatic caner growth and expansion.

In some embodiments, the disclosure provides a method of treating a pancreatic disease and/or hepatic disease in a subject in need thereof that comprises:
(a) administering an effective amount of a HIF1-α Pathway Inhibitor or a HIF1-α Inhibitor and a GPR81 inhibitor to the subject;
(b) administering an effective amount of a PFKFB3 inhibitor and a GPR81 inhibitor to the subject to the subject;
(c) administering an effective amount of a HIF1-α Pathway Inhibitor or a HIF1-α Inhibitor, a PFKFB3 inhibitor, and a GPR81 inhibitor to the subject;
(d) administering an effective amount of a GPR81 inhibitor Inhibitor to the subject, wherein the subject has previously been administered a PFKFB3 Inhibitor;
(e) administering an effective amount of a GPR81 inhibitor to the subject wherein the subject has previously been administered a HIF1-α Pathway Inhibitor or a HIF1-α Inhibitor; or
(f) administering an effective amount of a GPR81 inhibitor to the subject wherein the subject has previously been administered a HIF1-α Pathway Inhibitor or a HIF1-α Inhibitor and a PFKFB3 Inhibitor; and wherein the PFKFB3 inhibitor does not inhibit PI3K/AKT/mTOR pathway or HIF1-α.

The term “GPR81 Inhibitor” as used herein refers to a composition that inhibits or reduces GPR81 directly or indirectly via inhibiting one or more activities of the GPR81 signaling cascade.

The “GPR81 Inhibitors” that can be administered according to the provided methods are not particularly limited. In some embodiments, the administered GPR81 Inhibitor is an antibody or a GPR81-binding antibody fragment (e.g., a single chain antibody, a single-domain antibody (e.g., a VHH), a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, ENMD-1198, miRNA, dsRNA, ssRNA, and shRNA), a peptibody, a nanobody, or a small molecule.

In some embodiments, the GPR81 Inhibitor administered according to the provided methods has an IC50 for a GPR81 activity/function of 100 μM or lower concentration for a GPR81 activity. In some embodiments, the GPR81 Inhibitor has an IC50 of at least or at most or about 200, 100, 80, 50, 40, 20, 10, 5, or 1 M, or at least or at most or about 100, 10, or 1 nM, or lower (or any range or value derivable therefrom). In some embodiments, the GPR81 Inhibitor inhibits the expression of GPR81. Assays for determining the ability of a compound to inhibit GPR81 activity are known in the art. In some embodiments, the inhibition of GPR81 activity or expression is a decrease as compared with a control level or sample. The GPR81 Inhibitors that can be administered according to the provided methods are not particularly limited. In some embodiments, the GPR81 Inhibitor modulates one or more of GPR81 binding to lactate, GPR81 signal transduction, GPR81 mRNA expression; GPR81 protein translation or degradation.

In some embodiments, the GPR81 Inhibitor administered according to the provided methods is a small molecule. In some embodiments, the GPR81 Inhibitor administered according to the provided methods is a protein or polypeptide (e.g., an anti GPR81 antibody or antibody fragment that binds GPR81). In some embodiments, the GPR81 Inhibitor administered according to the provided methods is a therapeutic nucleic acid (e.g., an aptamer, antisense molecule, ribozyme, a Dicer substrate, siRNA, miRNA, dsRNA, ssRNA, or an shRNA).

Representative examples of human GPR81 coding sequences are provided in GenBank accession number NM_032554. The sequences associated with the each of these Genbank accession numbers is hereby incorporated by reference herein in its entirety for all purposes. Therapeutic nucleic acids that inhibit GPR81 activity can routinely be designed and prepared based on each of the above human GPR81 transcript sequences using methods known in the art.

### Kits for Administration of Active Agents

In another embodiment, the disclosure provides a kit containing a HIF1-α Pathway Inhibitor and a PFKFB3 inhibitor and/or other therapeutic and delivery agents. In some embodiments, a kit for preparing and/or administering a therapy described herein may be provided. The kit may comprise one or more sealed vials containing any of the pharmaceutical compositions, therapeutic agents and/or other therapeutic and delivery agents. In some embodiments, the kits comprise lipid delivery systems. In some embodiments, the lipid is in one vial, and the therapeutic agent is in a separate vial. The kit may include, for example, at least one inhibitor of PFKFB3 expression/activity, at least one inhibitor of HIF1-alpha expression/activity, and one or more reagents to prepare, formulate, and/or administer the components described herein or perform one or more steps of the methods. The some embodiments, the kit includes at least one inhibitor of PFKFB3 expression/activity, at least one inhibitor of HIF1-alpha expression/activity, at least one inhibitor of GPR81 expression/activity. In some embodiments, the kit may also comprise a suitable container means, which is a container that will not react with components of the kit, such as an eppendorf tube, an assay plate, a syringe, a bottle, or a tube. The container may be made from sterilizable materials such as plastic or glass.

The kit may further include an instruction sheet that outlines the procedural steps of the methods set forth herein, and will follow substantially the same procedures as described herein or are known to those of ordinary skill. The instruction information may be in a computer readable media containing machine-readable instructions that, when executed using a computer, cause the display of a real or virtual procedure of delivering a pharmaceutically effective amount of a therapeutic agent.

In some embodiments, kits may be provided to evaluate the expression of PFKFB3 and/or HIF-α or related molecules. Such kits can be prepared from readily available materials and reagents. For example, such kits can comprise any one or more of the following materials: enzymes, reaction tubes, buffers, detergent, primers and probes, nucleic acid amplification, and/or hybridization agents. In a particular embodiment, these kits allow a practitioner to obtain samples in blood, tears, semen, saliva, urine, tissue, serum, stool, colon, rectum, sputum, cerebrospinal fluid and supernatant from cell lysate. In another embodiment, these kits include the needed apparatus for performing RNA extraction, RT-PCR, and gel electrophoresis. Instructions for performing the assays can also be included in the kits.

Kits may comprise components, which may be individually packaged or placed in a container, such as a tube, bottle, vial, syringe, or other suitable container means. The components may include probes, primers, antibodies, arrays, negative and/or positive controls. Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

The kit can further comprise reagents for labeling PFKFB3 and/or HIF-1 alpha in the sample. The kit may also include labeling reagents, including at least one of amine-modified nucleotide, poly(A) polymerase, and poly(A) polymerase buffer. Labeling reagents can include an amine-reactive dye or any dye known in the art.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit (labeling reagent and label may be packaged together), the kit also will generally contain a second, third or other additional container into which the additional components may be separately placed. However, various combinations of components may be comprised in a vial. The kits may also include a means for containing the nucleic acids, antibodies or any other reagent containers in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly preferred. Alternatively, the components of the kit may be provided as dried powder(s). When reagents and/or components are provided as a dry powder, the powder can be reconstituted by the addition of a suitable solvent. It is envisioned that the solvent may also be provided in another container means. The container means will generally include at least one vial, test tube, flask, bottle, syringe and/or other container means, into which the nucleic acid formulations are placed, preferably, suitably allocated. The kits may also comprise a second container means for containing a sterile, pharmaceutically acceptable buffer and/or other diluent.

The kits may include a means for containing the vials in close confinement for commercial sale, such as, e.g., injection and/or blow-molded plastic containers into which the desired vials are retained. The kit may also include instructions for employing the kit components as well the use of any other reagent not included in the kit. Instructions may include variations that can be implemented.

### Methods of Administration

The regimen of administration according to a method provided herein (e.g., dose combined with frequency of administration) will generally involve administration in an amount and at a frequency to provide for a desired effect, e.g., administration of an amount effective to provide for improvement in one or more symptoms of a pancreatic disease and/or hepatic disease in a subject such as one or more symptoms associated with pancreatic disease and/or hepatic disease. Administration of each drug in combination may be by any suitable means that can be combined with other ingredients to alleviate the condition of the patient or cause concentration of the drug to effectively treat the disease or disorder. Possible compositions include those suitable for oral, rectal, topical (including transdermal, oral and sublingual), or parenteral (including subcutaneous, intramuscular, intravenous and intradermal) administration.

In some embodiments of the present invention, compositions are administered to a patient alone or in combination with other therapies, pharmaceuticals, supplements, and/or a specified diet, or in pharmaceutical compositions where it is mixed with excipient(s) or other pharmaceutically acceptable carriers. Depending on the goal of administration (e.g., severity of condition, duration of treatment, etc.), compositions (e.g., comprising a compound of Formula I, such as DMB) may be formulated and administered systemically or locally. Techniques for formulation and administration may be found in the latest edition of “Remington's Pharmaceutical Sciences” (Mack Publishing Co, Easton Pa.). Suitable routes may, for example, include oral or transmucosal administration; as well as parenteral delivery, including intramuscular, subcutaneous, intramedullary, intrathecal, intraventricular, intravenous, intraperitoneal, or intranasal administration. In some embodiments, a compound of Formula I (e.g., DMB) may be administered in the form of a solid, semi-solid or liquid dosage form: such as tablet, capsule, pill, powder, suppository, solution, elixir, syrup, suspension, cream, lozenge, paste and spray formulated appropriately to provide the desired therapeutic profile. As those skilled in the art would recognize, depending on the chosen route of administration, the composition form is selected.

The phrases “parenteral administration” and “administered parenterally” as used herein refer to modes of administration other than enteral and topical administration, such as injections, and include without limitation intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

The pharmaceutical compositions may be formulated according to conventional pharmaceutical practice (see, e.g., Remington: The Science and Practice of Pharmacy (20th ed.), Ed. A R Gennaro, Lippincott Williams & Wilkins, 2000 and Encyclopedia of Pharmaceutical Technology, eds J. Swarbrick and J C Boylan, 1988-1999, Marcel Dekker, New York).

Formulations suitable for oral administration can be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such formulations can be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture. For example, a tablet can be prepared by compressing or molding a powder or granule containing the active agent, optionally with one or more accessory ingredients. Compressed tablets can be prepared by compressing, in a suitable machine, the compound in a free-flowing form, such as a powder or granules optionally mixed with a binder, lubricant, inert diluent, and/or surface active/dispersing agent(s). Molded tablets can be made by molding, in a suitable machine, the powdered compound moistened with an inert liquid binder.

Formulations suitable for buccal (sub-lingual) administration include lozenges having the active agent in a flavored base, usually sucrose and acacia or tragacanth; and pastilles containing the active agent in an inert base such as gelatin and glycerin or sucrose and acacia.

Formulations for parenteral administration are conveniently sterile aqueous preparations of the active agent, which preparations are preferably isotonic with the blood of the intended recipient. These preparations can be administered by means of subcutaneous, intravenous, intramuscular, or intradermal injection. Such preparations can conveniently be prepared by admixing the compound with water or a glycine buffer and rendering the resulting solution sterile and isotonic with the blood.

Formulations suitable for topical application (e.g., in the oral passage, nasopharynx, or oropharynx) take the form of an ointment, cream, lotion, paste, gel, spray, aerosol, or oil. Carriers which can be used include vaseline, lanoline, polyethylene glycols, alcohols, transdermal enhancers, and combinations of two or more thereof.

In some embodiment, the disclosure provides a method of treatment wherein the compositions provided herein are administered in combination with one or more additional Therapeutic agent(s). The combination of the provided compositions and Therapeutic agent(s) may be administered or co-administered (e.g., consecutively, simultaneously, at different times) in any conventional dosage form. Co-administration in the present context refers to the administration of more than one Therapeutic agent to a subject in the course of a coordinated treatment to achieve an improved clinical outcome. Such co-administration may also be coextensive, that is, occurring during overlapping periods of time. For example, a first Therapeutic agent may be administered to a patient before, concomitantly, before and after, or after a second active agent is administered. In some embodiments the therapeutic agents are combined/formulated in a single composition and thus administered to the subject at the same time.

### Methods of Treatment and Use

### Pancreatic Steatosis

Obesity and metabolic syndrome (including obesity, hyperglycemia, dyslipidemia, hypertension and insulin resistance) lead to metabolic derangements that result in lipid mishandling by adipocytes and fat infiltration in the pancreas. Adipocytokine imbalances in circulation and in the pancreatic microenvironment cause chronic, low-grade inflammation and result in beta cell and acinar cell apoptosis that leads to pancreatic endocrine and exocrine dysfunction. Furthermore, these adipocytokines regulate cell growth, differentiation, as well as angiogenesis and lymphatic spread.

Pancreatic steatosis describes a disease ranging from infiltration of fat in the pancreas to pancreatic inflammation, and development of pancreatic fibrosis. The consequences of adipocyte infiltration in the pancreas are thought to initiate carcinogenesis, leading to pancreatic intraepithelial neoplasia and pancreatic ductal adenocarcinoma.

In some embodiments, the disclosure provides methods and compositions for treating pancreatic steatosis (PS).

In some embodiments, the disclosure provides methods and compositions for treating pancreatic steatosis in a subject in need thereof comprising:
(a) administering an effective amount of a HIF1-α Pathway Inhibitor and an PFKFB3 inhibitor to the subject;
(b) administering an effective amount of a HIF1-α Pathway Inhibitor to the subject, wherein the subject has previously been administered a PFKFB3 Inhibitor; or
(c) administering an effective amount of a PFKFB3 Inhibitor to the subject, wherein the subject has previously been administered a HIF1-α Pathway Inhibitor; and wherein the PFKFB3 inhibitor does not inhibit PI3K/AKT/mTOR pathway or HIF1-α.

In one embodiment, the subject is administered an effective amount of the HIF1-α Pathway Inhibitor and the PFKFB3 inhibitor. In one embodiment, the subject is administered an effective amount of the HIF1-α Pathway Inhibitor and the subject has previously been administered the PFKFB3 Inhibitor. In one embodiment, the subject is administered an effective amount of the PFKFB3 Inhibitor and the subject has previously been administered the HIF1-α Pathway Inhibitor.

The terms “pancreatic steatosis” and fatty pancreas are used interchangeably to refer to pancreatic fact accumulation.

In some embodiments, the PS treated according to the provided methods is nonalcoholic fatty pancreas disease. Treated nonalcoholic fatty pancreas diseases include, but are not limited to, nonalcoholic fatty steatopancreatitis, pancreatic lipomatosis, pancreatic steatosis, lipomatous pseudohypertrophy of pancreas, fatty replacement of pancreas, and fatty infiltration of pancreas.

In particular embodiments, the PS treated according to the provide methods is non-alcoholic fatty pancreas disease (NASP). In further embodiments, the treated PS is NAFPD non-alcoholic fatty pancreas disease that includes obesity and metabolic syndrome. Metabolic syndrome is a group of five conditions that can lead to heart disease, diabetes, stroke and other health problems. The conditions of metabolic syndrome include diabetes with insulin resistance, arterial hypertension, obesity, and dyslipidemia (hypertriglycidemia, decrease of HDL cholesterol).

In some embodiments the PS treated according to the provide methods is alcoholic fatty pancreas disease. Treated alcoholic fatty pancreas diseases include, but are not limited to, alcoholic fatty steatopancreatitis and alcoholic pancreatitis.

In some embodiments, the subject is at risk of having PS. In some embodiments, a method provided herein (e.g., any of (a)-(c) above), is performed as a prophylactic treatment for PS.

In some embodiments, the provided methods and compositions prevent PS in a subject at risk for developing PS, e.g., a subject having one or more risk factors associated with development of PS. In some embodiments, the subject has one or more risk factors selected from: obesity, metabolic syndrome, arterial hypertension, hypertriglyceridemia, the changes of HDL cholesterol, diabetes e.g., MD-T2), hepatic steatosis, NAFLD, malnutrition, taking certain medications such as rosiglitazone, corticosteroids, octreotide, and gemcitabine, hemochromatosis, infections (viral infection with Reovirus), chronic hepatitis B infection, having or having a family history of a congenital disease (e.g., Shwachman-Diamond syndrome, Johanson-Blizzard syndrome, cystic fibrosis, heterozygous carboxyl-ester lipase mutation), alcohol abuse (excessive alcohol consumption), having necrotizing pancreatitis, having recurrent acute pancreatitis, and having a family history of chronic pancreatitis. In particular embodiments, the risk factor is obesity or obesity as part of metabolic syndrome.

In some embodiments, the disclosure provides methods and compositions that prevent, inhibit or delay the onset of PS by administration of the provided compositions to a subject before the onset of PS, e.g., before the onset of one or more symptoms of PS.

In some embodiments, the HIF1-α Pathway Inhibitor and the PFKFB3 inhibitor are administered before the onset of one or more symptoms of PS. In some embodiments, the provided methods prevent PS. In some embodiments, the provided methods delay the onset of PS. In some embodiments, the provided methods are administered to a subject at risk for developing PS. In such subjects, prevention of PS may be monitored by lack of typical hallmarks of PS. For example, subjects to whom an effective amount of a HIF1-alpha inhibitor and PFKFB3 inhibitor is administered prophylactically may not experience or may experience a reduced incidence of one or more of the following symptoms: fatty content in the pancreas of more than 25%, abdominal pain (or a feeling of fullness in the upper right side of the abdomen); nausea, loss of appetite or weight loss; jaundice (yellowish skin and/or whites of the eyes); edema (swollen abdomen and/or legs); extreme tiredness or mental confusion; and weakness.

In some embodiments, the subject has been diagnosed as having PS. A spectrum of suitable methods for routinely diagnosing and monitoring PS are generally known in the art and include MRI proton density fat fraction, computed tomography, endoscopic ultrasound, ultrasound elastography, and transabdominal ultrasonic examination that compares pancreatic parenchymatous echogenity versus renal or hepatic echogenity.

In some embodiments, the HIF1-α Pathway Inhibitor and the PFKFB3 inhibitor are administered after the onset of one or more symptoms of PS. In some embodiments, the subject exhibits at least one of the following: fatty content in the pancreas of at 25% or more, abdominal pain (or a feeling of fullness in the upper right side of the abdomen); nausea, loss of appetite or weight loss; jaundice (yellowish skin and/or whites of the eyes); edema (swollen abdomen and/or legs); extreme tiredness or mental confusion; and weakness. In some embodiments, the provided methods and compositions may reduce the incidence, severity, or level of one or more of the above symptoms.

In some embodiments, the HIF1-α Pathway Inhibitor administered according to a method provided herein is an antibody or antigen-binding fragment thereof (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α Pathway binding polypeptide, or a small molecule HIF1-α Pathway Inhibitor.

In some embodiments, the administered HIF1-α Pathway Inhibitor is silibinin, PX-478 or YC-1, or a salt thereof.

In some embodiments the administered HIF1-α Pathway Inhibitor is ganetespib (ST-9090), phenethyl isothocyanate, or BAY-87-2243, or a salt thereof.

In some embodiments, the HIF1-α Pathway Inhibitor administered according to a method provided herein is a HIF1-α Inhibitor. In some embodiments, the HIF1-α Inhibitor does not inhibit the PI3K/AKT/mTOR pathway. In some embodiments, the HIF1-α Inhibitor is an antibody or antigen-binding fragment thereof (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α binding polypeptide, or a small molecule HIF1-α Inhibitor.

In some embodiments, the administered HIF1-α Inhibitor is antisense oligonucleotide EZN-2968 or nanobody AG-1, AG-2, AG-3, AG-4, AG-5, VHH212, or AHPC.

In some embodiments, the PFKFB3 Inhibitor administered according to a method provided herein is an antibody or antigen-binding antibody fragment (e.g., a single chain antibody, a single-domain antibody, a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a PFKFB3 binding polypeptide, or a small molecule PFKFB3 Inhibitor.

In some embodiments, the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof.

In some embodiments, the administered PFKFB3 Inhibitor is KAN0436151 or KAN0436067, or a salt thereof.

In some embodiments, the administered PFKFB3 inhibitor is AZ67, or a salt thereof.

In some embodiments, the administered PFKFB3 inhibitor has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof. In some embodiments, the administered PFKFB3 inhibitor has the structure of formula AZ44-AZ70 or AZ71, depicted in FIG. 1E, or a salt thereof.

In some embodiments, a method provided herein for treating PS is performed by co-administering the HIF1-α Pathway Inhibitor and the PFKFB3 inhibitor to the subject.

In some embodiments, the administration of the HIF1-α Pathway Inhibitor and/or the PFKFB3 inhibitor is administered orally. In some embodiments, the administration of the HIF1-α Pathway Inhibitor and/or the PFKFB3 inhibitor is administered, via transmucosal administration, syrup, topical administration, parenteral administration, injection, subdermal administration, rectal administration, buccal administration or transdermal administration.

In some embodiments, treating PS according to a method provided herein comprises reducing one or more symptoms of PS in the subject compared to a control subject or compared to the subject prior to treatment with the HIF1-α Pathway Inhibitor and the PFKFB3 inhibitor.

In some embodiments, the provided methods result in reduction of pancreatic fat (e.g., steatosis); reduced desmoplasia, e.g., in adipose tissues and/or the pancreas; improved function of adipocytes and/or pancreas; reduced hypoxia in adipose tissue and/or pancreas; reduced inflammation of adipose tissue and/or pancreas; reduced pancreatitis; and/or reduced pancreatic lesions caused by fatty pancreas. In some embodiments, the provided methods result in a reduction of PS biomarkers (e.g., associated with angiotensin II (AngII) type-1 receptor (ATI) signaling and/or pro-inflammatory cytokines, e.g., IL-1 beta. In some embodiments, the one or more symptoms of PS are reduced by at least 10%, at least 20%, at least 30%, at least 40%, or at least 50% compared to the control subject or compared to the subject prior to treatment with the HIF1-α Pathway Inhibitor and the PFKFB3 inhibitor.

In some embodiments, the pancreatic fat of the subject is reduced by at least 10%, at least 20%, at least 30%, at least 40%, or at least 50% compared to in the subject prior to treatment with the HIF1-α Pathway Inhibitor and the PFKFB3 inhibitor.

In some embodiments, the inflammation of adipose tissue and/or inflammation of the pancreas is reduced by at least 10%, at least 20%, at least 30%, at least 40%, or at least 50%, compared to in the subject prior to treatment with the HIF1-α Pathway Inhibitor and the PFKFB3 inhibitor.

In some embodiments, the pancreatitis is reduced by at least 10%, at least 20%, at least 30%, at least 40%, or at least 50%, compared to in the subject prior to treatment with the HIF1-α Pathway Inhibitor and the PFKFB3 inhibitor.

In additional embodiments, the provided methods include further administering an additional therapeutic agent to the subject.

### Pancreatic Cancer

In some embodiments, the disclosure provides methods and compositions for treating Pancreatic cancer. Pancreatic cancer is a malignant growth of the pancreas that mainly occurs in the cells of the pancreatic ducts. This disease is the ninth most common form of cancer, yet it is the fourth and fifth leading cause of cancer death in men and women, respectively. Cancer of the pancreas is almost always fatal, with a five-year survival rate that is less than 3%.

In some embodiments, the disclosure provides methods and compositions for treating pancreatic cancer in a subject in need thereof comprising:
(a) administering an effective amount of a HIF1-α Pathway Inhibitor and an PFKFB3 inhibitor to the subject;
(b) administering an effective amount of a HIF1-α Pathway Inhibitor to the subject, wherein the subject has previously been administered a PFKFB3 Inhibitor; or
(c) administering an effective amount of a PFKFB3 Inhibitor to the subject, wherein the subject has previously been administered a HIF1-α Pathway Inhibitor; and wherein the PFKFB3 inhibitor does not inhibit PI3K/AKT/mTOR pathway or HIF1-α.

In one embodiment, the subject is administered an effective amount of the HIF1-α Pathway Inhibitor and the PFKFB3 inhibitor. In one embodiment, the subject is administered an effective amount of the HIF1-α Pathway Inhibitor and the subject has previously been administered the PFKFB3 Inhibitor. In one embodiment, the subject is administered an effective amount of the PFKFB3 Inhibitor and the subject has previously been administered the HIF1-α Pathway Inhibitor.

In some embodiments, the pancreatic cancer treated according to the provided methods is an exocrine pancreatic cancer or a neuroendocrine pancreatic cancer.

In some embodiments, the pancreatic cancer is an exocrine pancreatic cancer. In some embodiments, the pancreatic cancer is an exocrine pancreatic cancer selected from: an adenocarcinoma, a squamous cell carcinoma, a adenosquamous carcinoma and a colloid carcinoma. In some embodiments, the pancreatic cancer is a squamous cell carcinoma, adenosquamous carcinoma, or a colloid carcinoma. In particular embodiments, the pancreatic cancer is a ductal adenocarcinoma (PDAC).

In some embodiments, the pancreatic cancer is a neuroendocrine pancreatic cancer.

In some embodiments, the subject is at risk of having pancreatic cancer (e.g., PDAC). In some embodiments, a method provided herein (e.g., any of (a)-(c) above), is performed as a prophylactic treatment for pancreatic cancer.

In some embodiments, the provided methods and compositions prevent pancreatic cancer (e.g., PDAC) in a subject at risk for developing pancreatic cancer, e.g., a subject having one or more risk factors associated with development of pancreatic cancer. In some embodiments, the subject has one or more risk factors selected from: being older than 40, smoking, excessive alcohol consumption, having diabetes (e.g., type 2 diabetes), developing diabetes at an older age, being obese, having chronic pancreatitis, having fatty pancreas, having Nonalcoholic fatty liver disease (NAFLD), being male, having a family history of chronic pancreatitis, pancreatic cancer, or BRCA related cancer (e.g., breast cancer); being of African American or Ashkenazi Jewish descent, and being exposed to chemicals used by dry cleaners and metal workers.

In some embodiments, the disclosure provides methods and compositions that prevent, inhibit or delay the onset of pancreatic cancer (e.g., PDAC) by administration of the provided compositions to a subject before the onset of pancreatic cancer, e.g., before the onset of one or more symptoms of pancreatic cancer.

In some embodiments, the HIF1-α Pathway Inhibitor and the PFKFB3 inhibitor are administered before the onset of one or more symptoms of pancreatic cancer (e.g., PDAC). In some embodiments, the provided methods prevent pancreatic cancer. In some embodiments, the provided methods delay the onset of pancreatic cancer (e.g., PDAC)

In some embodiments, the provided methods are administered to a subject at risk for developing pancreatic cancer. In such subjects, prevention of pancreatic cancer may be monitored by lack of typical hallmarks of pancreatic cancer. For example, subjects to whom an effective amount of a HIF1-alpha inhibitor and PFKFB3 inhibitor is administered prophylactically may not experience or may experience a reduced incidence of one or more of the following symptoms: pancreatitis, jaundice, or the yellowing of the eyes and skin; lack of appetite; abdominal pain; change in stool; enlarged gallbladder; sudden unexplained weight loss; itchy skin, palms; diabetes, and a change in taste.

In some embodiments, biological samples from the subject do not have a level of a pancreatic cancer biomarker (e.g., CA19-9) that is consistent with pancreatic cancer. the level of a pancreatic cancer biomarker in the subject's serum does not have elevated levels of 1 or more pancreatic cancer biomarkers (e.g., creatine kinase (CK-MB), troponin, N-terminal pro B-type natriuretic peptide, alpha-1 antitrypsin, C-reactive protein, apolipoprotein A1, apolipoprotein B, creatinine, alkaline phosphatase, and transferrin).

In some embodiments, the subject has been diagnosed as having pancreatic cancer. Current methods for diagnosing and monitoring pancreatic cancer generally include clinical symptoms, electrocardiography (ECG), and measurement of peripheral circulation biomarkers. Angiography is also used for severe chest pain usually associated with unstable angina and acute fatty pancreas (APS). Patients with pancreatic cancer often have constricted chest pain that often spreads inside the neck, chin, shoulders, or left or both arms, and may be accompanied by symptoms of dyspnea, sweating, palpitation, head wandering, and nausea. Myocardial ischemia can cause changes in the diagnostic ECG, such as changes in the Q wave and ST segment. Elevated plasma concentrations of cardiac enzymes of the subject reflect the degree of cardiac tissue necrosis associated with severe unstable angina and fatty pancreas.

In some embodiments, the HIF1-α Pathway Inhibitor and the PFKFB3 inhibitor are administered after the onset of one or more symptoms of pancreatic cancer. In some embodiments, the subject exhibits at least one of the following: pancreatitis, jaundice, or the yellowing of the eyes and skin; lack of appetite; abdominal pain; change in stool; enlarged gallbladder; sudden unexplained weight loss; itchy skin, palms; diabetes, and a change in taste. In some embodiments, the provided methods and compositions may reduce the incidence, severity, or level of one or more of the above symptoms.

In one embodiment, the pancreatic cancer is Stage I, II, III or IV.

In a particular embodiment, the pancreatic cancer is an exocrine pancreatic cancer selected from the group consisting of pancreatic ductal adenocarcinoma (PDAC), adenosquamous carcinoma, squamous cell carcinoma, giant cell carcinoma, acinar cell carcinoma and small cell carcinoma.

In a particular embodiment, the pancreatic cancer is a ductal adenocarcinoma, e.g., resectable pancreatic ductal adenocarcinoma (PDAC), which arises within the exocrine component of the pancreas. As used herein, “adenocarcinoma” refers to a cancerous tumor as opposed to an “adenoma” which refers to a benign (non-cancerous) tumor made up of cells that form glands (collections of cells surrounding an empty space). As used herein, “pancreatic ductal adenocarcinoma cell” refers to a cancerous cell that had the capability to form or originated from the ductal lining of the pancreas. A pancreatic ductal adenocarcinoma cell may be found within the pancreas forming a gland, or found within any organ as a metastasized cell or found within the blood stream of lymphatic system. As used herein, “ductal cell”, in reference to a pancreas, refers to any cell that forms or has the capability to form or originated from the ductal lining of ducts within and exiting from the pancreas.

In another embodiment, the pancreatic cancer is a pancreatic endocrine tumor, also known as islet cell tumors, pancreas endocrine tumors (PETs) and pancreatic neuroendocrine tumors (PNETs), which arises from islet cells. In a particular embodiment, the pancreatic cancer is an endocrine pancreatic cancer selected from the group consisting of insulinomas (i.e., arising from insulin-producing cells), glucagonomas (i.e., arising from glucagon-producing cells), somatostatinomas (i.e., arising from somatostatin-making cells), gastrinomas (i.e., arising from a gastrin-producing cells), VlPomas (arising from vasoactive intestinal peptide-making cells) and non-secreting islet tumors of the pancreas.

In one embodiment, the subject suffers from one or more of: hepatocellular carcinoma, non-resectable pancreatic cancer, locally advanced pancreatic cancer, borderline resectable pancreatic cancer, locally advanced pancreatic ductal adenocarcinoma, borderline resectable pancreatic ductal adenocarcinoma, metastatic pancreatic cancer, chemotherapy-resistant pancreatic cancer, pancreatic ductal adenocarcinoma, squamous pancreatic cancer, pancreatic progenitor, immunogenic pancreatic cancer, aberrantly differentiated endocrine exocrine (ADEX) tumors, an exocrine pancreatic cancer, pancreatic intraepithelial neoplasia, intraductal papillary mucinous neoplasms, mucinous cystic neoplasms, mucinous pancreas cancer, adenosquamous carcinoma, signet ring cell carcinoma, hepatoid carcinoma, colloid carcinoma, undifferentiated carcinoma, undifferentiated carcinomas with osteoclast-like giant cells, a pancreatic cystic neoplasm, an islet cell tumor, a pancreas endocrine tumor, or a pancreatic neuroendocrine tumor.

In some embodiments, the HIF1-α Pathway Inhibitor administered according to a method provided herein is an antibody or antigen-binding fragment thereof (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α Pathway binding polypeptide, or a small molecule HIF1-α Pathway Inhibitor.

In some embodiments, the administered HIF1-α Pathway Inhibitor is silibinin, PX-478 or YC-1, or a salt thereof.

In some embodiments the administered HIF1-α Pathway Inhibitor is ganetespib (ST-9090), phenethyl isothocyanate, or BAY-87-2243, or a salt thereof.

In some embodiments, the HIF1-α Pathway Inhibitor administered according to a method provided herein is a HIF1-α Inhibitor. In some embodiments, the HIF1-α Inhibitor does not inhibit the PI3K/AKT/mTOR pathway. In some embodiments, the HIF1-α Inhibitor is an antibody or antigen-binding fragment thereof (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α binding polypeptide, or a small molecule HIF1-α Inhibitor.

In some embodiments, the administered HIF1-α Inhibitor is antisense oligonucleotide EZN-2968 or nanobody AG-1, AG-2, AG-3, AG-4, AG-5, VHH212, or AHPC.

In some embodiments, the PFKFB3 Inhibitor administered according to a method provided herein is an antibody or antigen-binding antibody fragment (e.g., a single chain antibody, a single-domain antibody, a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a PFKFB3 binding polypeptide, or a small molecule PFKFB3 Inhibitor.

In some embodiments, the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof.

In some embodiments, the administered PFKFB3 Inhibitor is KAN0436151 or KAN0436067, or a salt thereof.

In some embodiments, the administered PFKFB3 inhibitor is AZ67, or a salt thereof.

In some embodiments, the administered PFKFB3 inhibitor has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof. In some embodiments, the administered PFKFB3 inhibitor has the structure of formula AZ44-AZ70 or AZ71, depicted in FIG. 1E, or a salt thereof.

In some embodiments, a method provided herein for treating pancreatic cancer is performed by co-administering the HIF1-α Pathway Inhibitor and the PFKFB3 inhibitor to the subject.

In some embodiments, the administration of the HIF1-α Pathway Inhibitor and/or the PFKFB3 inhibitor is administered orally. In some embodiments, the administration of the HIF1-α Pathway Inhibitor and/or the PFKFB3 inhibitor is administered, via transmucosal administration, syrup, topical administration, parenteral administration, injection, subdermal administration, rectal administration, buccal administration or transdermal administration.

In some embodiments, treating pancreatic cancer according to a method provided herein comprises reducing one or more symptoms of pancreatic cancer in the subject compared to a control subject or compared to the subject prior to treatment with the HIF1-α Pathway Inhibitor and the PFKFB3 inhibitor. In some embodiments, the provided methods result in one or more of: a decrease in tumor size, a suppression or decrease in tumor growth, no new tumor formation, a decrease in new tumor formation, an increase in survival or progression-free survival, no metastases, an increase in treatment options, delay in time from surgery to recurrence, reduction in jaundice, suppression of spread to liver, reduction in pain, improved appetite, improved digestion, reduction of gallbladder size, and reduced incidence of blood clots.

In some embodiments, the provided methods result in a normalized ECG (e.g., reversion of changes in the Q wave and ST segment to normal) or reduced levels of plasma concentrations of cardiac enzymes or other biomarkers (e.g., creatine kinase (CK-MB), troponin, N-terminal pro B-type natriuretic peptide, alpha-1 antitrypsin, C-reactive protein, apolipoprotein A1, apolipoprotein B, creatinine, alkaline phosphatase, and transferrin). In some embodiments, the one or more symptoms of pancreatic cancer are reduced by at least 10%, at least 20%, at least 30%, at least 40%, or at least 50% compared to the control subject or compared to the subject prior to treatment with the HIF1-α Pathway Inhibitor and the PFKFB3 inhibitor.

In some embodiments, 1, 2, 3, 4, 5, or more pancreatic cancer biomarkers (e.g., creatine kinase (CK-MB), troponin, N-terminal pro B-type natriuretic peptide, alpha-1 antitrypsin, C-reactive protein, apolipoprotein A1, apolipoprotein B, creatinine, alkaline phosphatase, and transferrin) in a biological sample of the subject is reduced by at least 10%, at least 20%, at least 30%, at least 40%, or at least 50% compared to a control subject or compared to the subject prior to treatment with the HIF1-α Pathway Inhibitor and the PFKFB3 inhibitor.

In one embodiment, the subject achieves a complete response. In one embodiment, the subject achieves a partial response. In one embodiment, the subject achieves stable disease. In one embodiment, the subject achieves a slower progressive disease.

In additional embodiments, the provided methods include further administering an additional therapeutic agent to the subject.

### Non-Alcoholic Fatty Liver Disease (NAFLD)

Fatty liver disease is a chronic condition characterized by excessive hepatic triglyceride deposition. It can be due to multiple causes; the major two forms being related to immoderate alcohol consumption or metabolic dysregulation in the absence of excessive alcohol intake. The latter has been termed non-alcoholic fatty liver disease (NAFLD).

NAFLD is characterized by fatty change of hepatocytes, accompanied by intralobular inflammation and fibrosis and it is commonly linked to the metabolic syndrome and its individual components (obesity, type 2 diabetes mellitus, dyslipidemia and hypertension).

The spectrum of NAFLD ranges from isolated hepatic steatosis also referred to as non-alcoholic fatty liver (NAFL) through non-alcoholic steatohepatitis (NASH). NASH is a common, often “silent” liver disease. Three major features characterize NASH and distinguish it from other liver disease of metabolic origin: abnormal fat accumulation or deposition in the liver (liver steatosis), liver inflammation, and liver injury or hepatic tissue damage (fibrosis). NASH is a potentially serious condition that carries a substantial risk of progression to end-stage liver disease, cirrhosis and hepatocellular carcinoma. Some patients who develop cirrhosis are at risk of liver failure and may eventually require a liver transplant.

In some embodiments, the disclosure provides methods and compositions for treating Non-alcoholic fatty liver disease (NAFLD).

In some embodiments, the disclosure provides methods and compositions for treating NAFLD in a subject in need thereof comprising:
(a) administering an effective amount of a HIF1-α Pathway Inhibitor and an PFKFB3 inhibitor to the subject;
(b) administering an effective amount of a HIF1-α Pathway Inhibitor to the subject, wherein the subject has previously been administered a PFKFB3 Inhibitor; or
(c) administering an effective amount of a PFKFB3 Inhibitor to the subject, wherein the subject has previously been administered a HIF1-α Pathway Inhibitor; and wherein the PFKFB3 inhibitor does not inhibit PI3K/AKT/mTOR pathway or HIF1-α.

In one embodiment, the subject is administered an effective amount of the HIF1-α Pathway Inhibitor and the PFKFB3 inhibitor. In one embodiment, the subject is administered an effective amount of the HIF1-α Pathway Inhibitor and the subject has previously been administered the PFKFB3 Inhibitor. In one embodiment, the subject is administered an effective amount of the PFKFB3 Inhibitor and the subject has previously been administered the HIF1-α Pathway Inhibitor.

In some embodiments, the NAFLD treated according to the provided methods is non-alcoholic fatty liver (NAFL). In some embodiments, the NAFLD treated according to the provided methods is non-alcoholic steatohepatitis (NASH). In some embodiments, the NAFLD treated according to the provided methods is NAFLD-associated liver fibrosis.

In some embodiments, the subject is at risk of having NAFLD. In some embodiments, a method provided herein (e.g., any of (a)-(c) above), is performed as a prophylactic treatment for NAFLD. In some embodiments, the subject is at risk of having NAFLD. In some embodiments, the method provided herein (e.g., any of (a)-(c) above), is performed as a prophylactic treatment for NASH. In some embodiments, the method provided herein (e.g., any of (a)-(c) above), is performed as a prophylactic treatment for NAFLD-associated liver fibrosis.

In some embodiments, the provided methods and compositions prevent NAFLD in a subject at risk for developing NAFLD, e.g., a subject having one or more risk factors associated with development of NAFLD. In some embodiments, the subject has one or more risk factors selected from: obesity, metabolic syndrome, hypertriglyceridemia, Type 2 diabetes, sleep apnea, hypothyroidism, hypopituitarism, polycystic ovary syndrome. NASH is more likely to occur: in older subjects, in subjects with diabetes, and in subjects with body fat concentrated in the abdomen.

In some embodiments, the subject treated according to a method provided herein is also suffering from type II diabetes mellitus, type I diabetes mellitus, pre-diabetes, insulin resistance, or obesity, wherein obesity is defined as the patient having a body mass index of ≥30.

In some embodiments, the disclosure provides methods and compositions that prevent, inhibit or delay the onset of NAFL, NASH, and/or NAFLD-associated liver fibrosis by administration of the provided compositions to a subject before the onset of NAFLD NASH, and/or NAFLD-associated liver fibrosis.

In some embodiments, the disclosure provides methods and compositions that prevent, inhibit or delay the onset of NAFL.

In some embodiments, the disclosure provides methods and compositions that prevent, inhibit or delay the onset of NASH. The preferred means in the art for distinguishing NASH from steatosis with inflammation is currently histological evaluation of liver biopsies using the so-called “NAFLD activity score” or “NAS)” (Kleiner et al., Hepatology 2005, vol. 41, pp. 1313-1321). Other scoring systems may also be used to diagnose NAFLD and the severity of its components. The NAS. The NAS is defined as the unweighted sum of the subscores for (i) steatosis, (ii) lobular inflammation and (iii) hepatocellular ballooning and specifically includes features of active injury that are potentially reversible in the short term. Each score is graded semi-quantitatively as described in the following table:

TABLE 1

| | | |
|---|---|---|
| Subscore | | |
| Histological | | |
| Feature | Definition | Score |

steatosis (0-3)

| | | |
|---|---|---|
| Grade | low- to medium-power evaluation of parenchymal | |
| | involvement by steatosis (% of cells) | |
| | <5% | 0 |
| | 5%-33% | 1 |
| | >33%-66% | 2 |
| | >66% | 3 |

lobular inflammation (0-3)

| | |
|---|---|
| overall assessment of all inflammatory foci | |
| no foci | 0 |
| 1 to <2 foci per 200* field | 1 |

Using the NRA, a subject is diagnosed, or considered to be suffering from NAFLD according to the present disclosure, if at least the score for steatosis (the “steatosis score”) is 1. NASH can be distinguished from NAFL or simple steatosis by the presence of hepatocyte ballooning (the “ballooning score”) with or without some degree of inflammation (the “inflammation score”). A NAS of <3 corresponds to NAFLD, 3-4 corresponds to borderline NASH, and >5 corresponds to NASH. This definition was used for the diagnostic algorithm presented in Table 2.

TABLE 2

| | (hepatocycle) | (lobular) | |
|---|---|---|---|
| steatosis | ballooning | inflammation | diagnosis |
| 0 | 0, 1, 2 | 0, 1, 2, 3 | No NAFLD |
| 1, 2, 3 | 0 | 0, 1. | NAFL |
| 1, 2, 3 | 0 | 2, 3 | NASH |
| 1, 2, 3 | 1 | 0, 1, 2, 3 | NASH |
| 1, 2, 3 | 2 | 0, 1, 2, 3 | NASH |

While the NAS determines the extent of NAFL and NASH (higher score means higher disease activity), the Kleiner fibrosis score can be used to determine the extent of fibrosis progression. Table 3 provides a definition of the Kleiner fibrosis score.

TABLE 3

| Fibrosis | comments | score |
|---|---|---|
| none | | 0 |
| portal/periportal | mild fibrosis | 1 |
| perisinusoidal and | moderate fibrosis between portal areas, but | 2 |
| portal/periportal | without destruction of the lobular structure | |
| bridging fibrosis | fibrotic bridging between portal areas and | 3 |
| | between portal areas and center veins | |
| cirrhosis | additionally pseudo-lobules formed | 4 |

In some embodiments, the HIF1-α Pathway Inhibitor and the PFKFB3 inhibitor are administered before the onset of one or more symptoms of NAFLD (e.g., NASH). In some embodiments, the provided methods prevent NAFLD. In some embodiments, the provided methods delay the onset of NAFLD. In some embodiments, the provided methods are administered to a subject at risk for developing NAFLD (e.g., NASH). In such subjects, prevention of NAFLD may be monitored by lack of typical hallmarks of NAFLD (e.g., NASH). For example, subjects to whom an effective amount of a HIF1-alpha inhibitor and PFKFB3 inhibitor is administered prophylactically may not experience or may experience a reduced incidence of one or more of the following symptoms (e.g., NASH).

In some embodiments, the HIF1-α Pathway Inhibitor and the PFKFB3 inhibitor are administered after the onset of one or more symptoms of NAFLD. In some embodiments, the subject exhibits at least one of the following: fatigue, pain or discomfort in the upper right abdomen, enlarged spleen, jaundice (yellowish skin and/or whites of the eyes); edema (swollen abdomen); easy bruising or bleeding; loss of appetite, and nausea. In some embodiments, the provided methods and compositions may reduce the incidence, severity, or level of one or more of the above symptoms.

In some embodiments, the HIF1-α Pathway Inhibitor administered according to a method provided herein is an antibody or antigen-binding fragment thereof (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α Pathway binding polypeptide, or a small molecule HIF1-α Pathway Inhibitor.

In some embodiments, the administered HIF1-α Pathway Inhibitor is silibinin, PX-478 or YC-1, or a salt thereof.

In some embodiments the administered HIF1-α Pathway Inhibitor is ganetespib (ST-9090), phenethyl isothocyanate, or BAY-87-2243, or a salt thereof.

In some embodiments, the HIF1-α Pathway Inhibitor administered according to a method provided herein is a HIF1-α Inhibitor. In some embodiments, the HIF1-α Inhibitor does not inhibit the PI3K/AKT/mTOR pathway. In some embodiments, the HIF1-α Inhibitor is an antibody or antigen-binding fragment thereof (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α binding polypeptide, or a small molecule HIF1-α Inhibitor.

In some embodiments, the administered HIF1-α Inhibitor is antisense oligonucleotide EZN-2968 or nanobody AG-1, AG-2, AG-3, AG-4, AG-5, VHH212, or AHPC.

In some embodiments, the PFKFB3 Inhibitor administered according to a method provided herein is an antibody or antigen-binding antibody fragment (e.g., a single chain antibody, a single-domain antibody, a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a PFKFB3 binding polypeptide, or a small molecule PFKFB3 Inhibitor.

In some embodiments, the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof.

In some embodiments, the administered PFKFB3 Inhibitor is KAN0436151 or KAN0436067, or a salt thereof.

In some embodiments, the administered PFKFB3 inhibitor is AZ67, or a salt thereof.

In some embodiments, the administered PFKFB3 inhibitor has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof. In some embodiments, the administered PFKFB3 inhibitor has the structure of formula AZ44-AZ70 or AZ71, depicted in FIG. 1E, or a salt thereof.

In some embodiments, a method provided herein for treating NAFLD is performed by co-administering the HIF1-α Pathway Inhibitor and the PFKFB3 inhibitor to the subject.

In some embodiments, the administration of the HIF1-α Pathway Inhibitor and/or the PFKFB3 inhibitor is administered orally. In some embodiments, the administration of the HIF1-α Pathway Inhibitor and/or the PFKFB3 inhibitor is administered, via transmucosal administration, syrup, topical administration, parenteral administration, injection, subdermal administration, rectal administration, buccal administration or transdermal administration.

In some embodiments, treating NAFLD according to a method provided herein comprises reducing one or more symptoms of NAFLD in the subject compared to a control subject or compared to the subject prior to treatment with the HIF1-α Pathway Inhibitor and the PFKFB3 inhibitor.

In some embodiments, the provided methods result in reduction of hepatic fat (e.g., steatosis); reduced fibrosis, e.g., in adipose tissues and/or the liver; improved function of adipocytes and/or liver; reduced hypoxia in adipose tissue and/or liver; reduced inflammation of adipose tissue and/or liver; reduced liver inflammation, reduction in alanine transaminase (ALT) and insulin levels, and/or reduced hepatic lesions caused by fatty liver. In some embodiments, the provided methods result in a reduction of NAFLD biomarkers (e.g., NAFLD biomarkers—increase in TNFα, IL-6, CRP, IL-1RA, PAI1, CXCL10, CK18, FGF21, and oxLDL and decrease in ADP and leptin; NASH biomarkers—increase in TNFα and IL-6 and decrease in ADP and leptin; fibrosis biomarkers—decrease in ADP and increase in leptin, hyaluronic acid, laminin, procollagen II, and TIMP1). In some embodiments, the one or more symptoms of NAFLD are reduced by at least 10%, at least 20%, at least 30%, at least 40%, or at least 50% compared to the control subject or compared to the subject prior to treatment with the HIF1-α Pathway Inhibitor and the PFKFB3 inhibitor.

In some embodiments, the provided methods result in a reduction of (a) steatosis, triglyceride content, inflammation, and/or apoptosis in liver tissue; (b) the serum aminotransferase level; (c) insulin-resistance, and optionally to improve glucose-tolerance in the subject.

In some embodiments, a provided method results in a lowering of NAS score in the subject by 1, 2, 3, or more points. In some embodiments, a provided method results in a subject having a NAS of <3.

In some embodiments, a provided method treats a subject suffering from NASH. Such subject can have NASH, could have been diagnosed with NASH, or could be genetically predisposed to the development of NASH, or could be predisposed to the development of NASH because he/she suffers from metabolic syndrome, obesity, diabetes or pre-diabetes. In still other embodiments a patient suffering from NASH is a patient that has been tested and found to display the clinical findings characteristic of NASH (abnormal accumulation of fat in the liver, liver inflammation and liver fibrosis), even though he or she may not show any physical symptoms of NASH yet. In some instances, a patient suffering from NASH displays symptoms of NASH even though a diagnosis has not yet been made.

In some embodiments, the provided methods result in slowing down or halting the progression of NASH into cirrhosis in the subject. In some embodiments, treatment results in the amelioration of at least one measurable physical symptom of NASH, such as, for example, weight loss, weakness or fatigue. In other embodiments, treatment results in amelioration of at least one clinical parameter or finding of NASH, such as, for example, abnormal liver fat accumulation, liver fibrosis as determined by biopsy, liver inflammation, abnormal levels of liver enzymes (e.g., ALT), abnormal levels of inflammatory cytokines or NAS score. In other embodiments, treatment results in the reduction, inhibition or slowing down of the progression of NASH, either physically by, e.g., stabilization of a measurable symptom or set of symptoms (such as fatigue, weight loss or weakness), or clinically/physiologically by, e.g., stabilization of a measurable parameter, such as abnormal fat accumulation in liver, abnormal levels of liver enzymes, abnormal levels of liver inflammatory markers, abnormal findings in a liver biopsy, NAS score or both. In another embodiment, treatment may also result in averting the cause and/or effects or clinical manifestation of NASH, or one of the symptoms developed as a result of NASH, prior to the disease or disorder fully manifesting itself. In some embodiments, treatment results in an increase in survival rate or survival time in a patient with NASH. In some embodiments, treatment results in the reduction of the potential for a patient with NASH needing a liver transplant. In other embodiments, treatment results in the elimination of the need for a NASH patient to undergo a liver transplant. In other embodiments, it results in the reduction of chances a patient with NASH will develop cirrhosis. In other embodiments, it results in prevention of progression to cirrhosis as determined by histology.

In some embodiments, the liver fat of the subject is reduced by at least 10%, at least 20%, at least 30%, at least 40%, or at least 50% compared to in the subject prior to treatment with the HIF1-α Pathway Inhibitor and the PFKFB3 inhibitor.

In some embodiments, the inflammation of adipose tissue and/or the liver is reduced by at least 10%, at least 20%, at least 30%, at least 40%, or at least 50%, compared to in the subject prior to treatment with the HIF1-α Pathway Inhibitor and the PFKFB3 inhibitor.

In some embodiments, the pancreatitis is reduced by at least 10%, at least 20%, at least 30%, at least 40%, or at least 50%, compared to in the subject prior to treatment with the HIF1-α Pathway Inhibitor and the PFKFB3 inhibitor.

In additional embodiments, the provided methods include further administering an additional therapeutic agent to the subject.

### Hepatocellular Cancer

In some embodiments, the disclosure provides methods and compositions for treating Hepatocellular carcinoma (HCC). Hepatocellular carcinoma develops due to malignant transformation of liver cells following chronic hepatitis or liver cirrhosis.

In some embodiments, the disclosure provides methods and compositions for treating Hepatocellular carcinoma (HCC) in a subject in need thereof comprising:
(a) administering an effective amount of a HIF1-α Pathway Inhibitor and an PFKFB3 inhibitor to the subject;
(b) administering an effective amount of a HIF1-α Pathway Inhibitor to the subject, wherein the subject has previously been administered a PFKFB3 Inhibitor; or
(c) administering an effective amount of a PFKFB3 Inhibitor to the subject, wherein the subject has previously been administered a HIF1-α Pathway Inhibitor; and wherein the PFKFB3 inhibitor does not inhibit PI3K/AKT/mTOR pathway or HIF1-α.

In one embodiment, the subject is administered an effective amount of the HIF1-α Pathway Inhibitor and the PFKFB3 inhibitor. In one embodiment, the subject is administered an effective amount of the HIF1-α Pathway Inhibitor and the subject has previously been administered the PFKFB3 Inhibitor. In one embodiment, the subject is administered an effective amount of the PFKFB3 Inhibitor and the subject has previously been administered the HIF1-α Pathway Inhibitor.

In some embodiments, the subject is at risk of having hepatocellular cancer. In some embodiments, a method provided herein (e.g., any of (a)-(c) above), is performed as a prophylactic treatment for hepatocellular cancer.

In some embodiments, the provided methods and compositions prevent hepatocellular cancer in a subject at risk for developing hepatocellular cancer, e.g., a subject having one or more risk factors associated with development of hepatocellular cancer. In some embodiments, the subject has one or more risk factors selected from: male, older than 55, chronic viral hepatitis (e.g., hepatitis B, hepatitis C), autoimmune hepatitis, NAFLD, NASH, fatty pancreas, smoking, excessive alcohol consumption, diabetes (e.g., type 2 diabetes), obese, having chronic pancreatitis, liver cirrhosis, primary biliary cirrhosis, family history of HCC, inherited metabolic diseases, hereditary hemochromatosis, alpha1-antitrypsin deficiency, Wilson's disease, and exposure to certain environmental factors such as aflatoxin.

In some embodiments, the disclosure provides methods and compositions that prevent, inhibit or delay the onset of hepatocellular cancer by administration of the provided compositions to a subject before the onset of hepatocellular cancer, e.g., before the onset of one or more symptoms of hepatocellular cancer.

Symptoms of HCC often do not appear in the early stages of cancer. Later, symptoms include weight loss, loss of appetite, fever, nausea, fatigue, upper abdominal pain, swelling in the abdomen and legs, yellowing of the skin (jaundice), easy bruising or bleeding, and liver failure.

In some embodiments, the HIF1-α Pathway Inhibitor and the PFKFB3 inhibitor are administered before the onset of one or more symptoms of hepatocellular cancer. In some embodiments, the provided methods prevent hepatocellular cancer. In some embodiments, the provided methods delay the onset of hepatocellular cancer.

In some embodiments, the provided methods are administered to a subject at risk for developing hepatocellular cancer. In such subjects, prevention of hepatocellular cancer may be monitored by lack of typical hallmarks of hepatocellular cancer. For example, subjects to whom an effective amount of a HIF1-alpha inhibitor and PFKFB3 inhibitor is administered prophylactically may not experience or may experience a reduced incidence of one or more of the following symptoms: weight loss, loss of appetite, fever, nausea, fatigue, upper abdominal pain, swelling in the abdomen and legs, yellowing of the skin (jaundice), easy bruising or bleeding, and liver failure.

In some embodiments, biological samples from the subject do not have a level of a hepatocellular cancer biomarker (e.g., EpCam, VEGF, EGFR, FLT1, theophylline, HCC-22-5, KRT23, AHSG, FTL, C16Cer, C16DHC, C18DHC, SiP, C24DHC, C24:1DHC, C18Cer, C20Cer, C24Cer, C24:1Cer, Sphingosine, and SA1P, CTSD, HYOU1, PSAP, and LAMP-2) that is consistent with hepatocellular cancer. The biological sample for analysis is typically blood, plasma, serum, mucous, tissue biopsy, tumor, ascites or cerebrospinal fluid from the patient. The sample can be analyzed for indication of neoplasia.

In some embodiments, the subject has been diagnosed as having hepatocellular cancer. Current methods for diagnosing and monitoring hepatocellular cancer generally include clinical symptoms, computerized axial tomography (CAT Scan), Magnetic Resonance Imaging (MRI), abdominal ultrasound, clotting tests, arteriography, or biopsy. Patients with hepatocellular cancer often have weight loss, loss of appetite, fever, nausea, fatigue, upper abdominal pain, swelling in the abdomen and legs, yellowing of the skin (jaundice), easy bruising or bleeding, and liver failure.

In some embodiments, the HIF1-α Pathway Inhibitor and the PFKFB3 inhibitor are administered after the onset of one or more symptoms of hepatocellular cancer. In some embodiments, the subject exhibits at least one of the following: weight loss, loss of appetite, fever, nausea, fatigue, upper abdominal pain, swelling in the abdomen and legs, yellowing of the skin (jaundice), easy bruising or bleeding, and liver failure. In some embodiments, the provided methods and compositions may reduce the incidence, severity, or level of one or more of the above symptoms.

In some of embodiments, the subject is genetically or otherwise predisposed (e.g., having a risk factor) to developing HCC

In one embodiment, the hepatocellular cancer is early stage HCC. In one embodiment, the hepatocellular cancer is non-metastatic HCC or primary HCC. In one embodiment, the hepatocellular cancer is advanced HCC, locally advanced HCC, or metastatic HCC. In one embodiment, the hepatocellular cancer is HCC in remission, or recurrent HCC. In some embodiments, the HCC is localized resectable, localized unresectable, or unresectable (i.e., the tumors involve all lobes of the liver and/or has spread to involve other organs (e.g., lung, lymph nodes, bone). In some embodiments, the HCC is, according to TNM classifications, a stage I tumor (single tumor without vascular invasion), a stage II tumor (single tumor with vascular invasion, or multiple tumors, none greater than 5 cm), a stage III tumor (multiple tumors, any greater than 5 cm, or tumors involving major branch of portal or hepatic veins), a stage IV tumor (tumors with direct invasion of adjacent organs other than the gallbladder, or perforation of visceral peritoneum), N1 tumor (regional lymph node metastasis), or M1 tumor (distant metastasis). In some embodiments, the HCC is, according to AJCC (American Joint Commission on Cancer) staging criteria, stage T1, T2, T3, or T4 HCC.

In one embodiment, the subject suffers from liver cell carcinomas, fibrolamellar variants of HCC, or a mixed hepatocellular cholangiocarcinoma.

In some embodiments, the HIF1-α Pathway Inhibitor administered according to a method provided herein is an antibody or antigen-binding fragment thereof (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α Pathway binding polypeptide, or a small molecule HIF1-α Pathway Inhibitor.

In some embodiments, the administered HIF1-α Pathway Inhibitor is silibinin, PX-478 or YC-1, or a salt thereof.

In some embodiments the administered HIF1-α Pathway Inhibitor is ganetespib (ST-9090), phenethyl isothocyanate, or BAY-87-2243, or a salt thereof.

In some embodiments, the HIF1-α Pathway Inhibitor administered according to a method provided herein is a HIF1-α Inhibitor. In some embodiments, the HIF1-α Inhibitor does not inhibit the PI3K/AKT/mTOR pathway. In some embodiments, the HIF1-α Inhibitor is an antibody or antigen-binding fragment thereof (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α binding polypeptide, or a small molecule HIF1-α Inhibitor.

In some embodiments, the administered HIF1-α Inhibitor is antisense oligonucleotide EZN-2968 or nanobody AG-1, AG-2, AG-3, AG-4, AG-5, VHH212, or AHPC.

In some embodiments, the PFKFB3 Inhibitor administered according to a method provided herein is an antibody or antigen-binding antibody fragment (e.g., a single chain antibody, a single-domain antibody, a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a PFKFB3 binding polypeptide, or a small molecule PFKFB3 Inhibitor.

In some embodiments, the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof.

In some embodiments, the administered PFKFB3 Inhibitor is KAN0436151 or KAN0436067, or a salt thereof.

In some embodiments, the administered PFKFB3 inhibitor is AZ67, or a salt thereof.

In some embodiments, the administered PFKFB3 inhibitor has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof. In some embodiments, the administered PFKFB3 inhibitor has the structure of formula AZ44-AZ70 or AZ71, depicted in FIG. 1E, or a salt thereof.

In some embodiments, a method provided herein for treating hepatocellular cancer is performed by co-administering the HIF1-α Pathway Inhibitor and the PFKFB3 inhibitor to the subject.

In some embodiments, the administration of the HIF1-α Pathway Inhibitor and/or the PFKFB3 inhibitor is administered orally. In some embodiments, the administration of the HIF1-α Pathway Inhibitor and/or the PFKFB3 inhibitor is administered, via transmucosal administration, syrup, topical administration, parenteral administration, injection, subdermal administration, rectal administration, buccal administration or transdermal administration.

In some embodiments, treating hepatocellular cancer according to a method provided herein comprises reducing one or more symptoms of hepatocellular cancer in the subject compared to a control subject or compared to the subject prior to treatment with the HIF1-α Pathway Inhibitor and the PFKFB3 inhibitor. In some embodiments, the provided methods result in one or more of: a decrease in the number of HCC cells, decrease in tumor size, a suppression or decrease in tumor growth, no new tumor formation, a decrease in new tumor formation, inhibition, retardation, slowing to some extent and preferably stop HCC cancer cell infiltration into peripheral organs; no metastases, an inhibition of tumor growth; prevention or delay occurrence and/or recurrence of tumor; relieve to some extent one or more of the symptoms associated with HCC; an increase in survival or progression-free survival, an/or increase in treatment options, delay in time from surgery to recurrence, reduction in jaundice, suppression of spread to liver, reduction in pain, improved appetite, improved digestion, reduction of gallbladder size, and reduced incidence of blood clots.

The terms “inhibiting,” “reducing,” “decreasing” with respect to tumor or cancer growth or progression refers to inhibiting the growth, spread, metastasis of a tumor or cancer in a subject by a measurable amount using any method known in the art. The growth, progression or spread of a tumor or cancer is inhibited, reduced or decreased if the tumor burden is at least about 10%, 20%, 30%, 50%, 80%, or 100% reduced, e.g.,

In some embodiments, the provided methods result in a HCC biomarker (e.g., EpCam, VEGF, EGFR, FLT1, theophylline, HCC-22-5, KRT23, AHSG, FTL, C16Cer, C16DHC, C18DHC, S1P, C24DHC, C24:1DHC, C18Cer, C20Cer, C24Cer, C24:1Cer, Sphingosine, and SA1P, CTSD, HYOU1, PSAP, and LAMP-2). In some embodiments, the one or more symptoms of hepatocellular cancer are reduced by at least 10%, at least 20%, at least 30%, at least 40%, or at least 50% compared to the control subject or compared to the subject prior to treatment with the HIF1-α Pathway Inhibitor and the PFKFB3 inhibitor.

In some embodiments, the level of 1, 2, 3, 4, 5, or more hepatocellular cancer biomarkers (e.g., EpCam, VEGF, EGFR, FLT1, theophylline, HCC-22-5, KRT23, AHSG, FTL, C16Cer, C16DHC, C18DHC, S1P, C24DHC, C24:1DHC, C18Cer, C20Cer, C24Cer, C24:1Cer, Sphingosine, and SA1P, CTSD, HYOU1, PSAP, and LAMP-2) in a biological sample of the subject is reduced by at least 10%, at least 20%, at least 30%, at least 40%, or at least 50% compared to a control subject or compared to the subject prior to treatment with the HIF1-α Pathway Inhibitor and the PFKFB3 inhibitor.

In one embodiment, the subject achieves a complete response. In one embodiment, the subject achieves a partial response. In one embodiment, the subject achieves stable disease. In one embodiment, the subject achieves a slower progressive disease.

In additional embodiments, the provided methods include further administering an additional therapeutic agent to the subject.

The disclosure of each of U.S. Appl. No. 63/189,204, U.S. Appl. No. 63/189,205, U.S. Appl. No. 63/189,206, and U.S. Appl. No. 63/189,207, each filed May 16, 2021, is herein incorporated by reference in its entirety. All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

What is claimed is:

1. A method of treating a pancreatic steatosis in a subject in need thereof comprising:
(a) administering an effective amount of a HIF1-α Pathway Inhibitor and an PFKFB3 inhibitor to the subject;
(b) administering an effective amount of a HIF1-α Pathway Inhibitor to the subject, wherein the subject has previously been administered a PFKFB3 Inhibitor; or
(c) administering an effective amount of a PFKFB3 Inhibitor to the subject, wherein the subject has previously been administered a HIF1-α Pathway Inhibitor;
wherein the PFKFB3 inhibitor does not inhibit PI3K/AKT/mTOR pathway or HIF1-α.

2. The method of claim 1, wherein the subject is administered an effective amount of the HIF1-α Pathway Inhibitor and the PFKFB3 inhibitor.

3. The method of claim 1, wherein the subject is administered an effective amount of the HIF1-α Pathway Inhibitor and wherein the subject has previously been administered the PFKFB3 Inhibitor.

4. The method of claim 1, wherein the subject is administered an effective amount of the PFKFB3 Inhibitor and wherein the subject has previously been administered the HIF1-α Pathway Inhibitor.

5. The method of any one of claims 1-4, wherein the method of any one of claim 1(a)-1(c) is administered as a prophylactic treatment for the pancreatic steatosis.

6. The method of any one of claims 1-4, wherein the subject has or is at risk of having the pancreatic steatosis.

7. The method of any one of claims 1-4, wherein the subject has or has been diagnosed as having the pancreatic steatosis.

8. The method of any one of claims 1-7, wherein the pancreatic steatosis is associated with infiltration of fat in the pancreas, infiltration of fat in the pancreas with pancreatic inflammation, or infiltration of fat in the pancreas with development of pancreatic fibrosis.

9. The method of any one of claims 1-8, wherein the pancreatic steatosis is associated with infiltration of fat in the pancreas.

10. The method of any one of claims 1-8, wherein the pancreatic steatosis is associated with infiltration of fat in the pancreas with pancreatic inflammation.

11. The method of any one of claims 1-8, wherein the pancreatic steatosis is associated with infiltration of fat in the pancreas with development of pancreatic fibrosis.

12. The method of any one of claims 1-8, wherein the pancreatic steatosis is associated with infiltration of fat in the pancreas with pancreatic inflammation with development of pancreatic fibrosis.

13. The method of any one of claims 1-12 wherein the administered HIF1-α Pathway Inhibitor is an antibody or antigen-binding antibody fragment (e.g., a single chain antibody, a single-domain antibody (e.g., a VHH), a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, MiRNA, dsRNA, ssRNA, and shRNA), a peptibody, a nanobody, a HIF1-α Pathway binding polypeptide, or a small molecule HIF1-α Pathway Inhibitor.

14. The method of any one of claims 1-13 wherein the administered HIF1-α Pathway Inhibitor is silibinin, PX-478 or YC-1, or a salt thereof.

15. The method of any one of claims 1-14 wherein the administered HIF1-α Pathway Inhibitor is ganetespib (ST-9090), phenethyl isothocyanate, or BAY-87-2243, or a salt thereof.

16. The method of any one of claims 1-15 wherein the administered HIF1-α Pathway Inhibitor is a HIF1-α Inhibitor.

17. The method of claim 16 wherein the HIF1-α Inhibitor is an antibody or antigen-binding antibody fragment (e.g., a single chain antibody, a single-domain antibody (e.g., a VHH), a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α binding polypeptide, or a small molecule HIF1-α Inhibitor.

18. The method of claim 16 or 17, wherein the administered HIF1-α Inhibitor is antisense oligonucleotide EZN-2968 or nanobody AG-1, AG-2, AG-3, AG-4, AG-5, VHH212, or AHPC.

19. The method of any one of claims 1-18, wherein the administered PFKFB3 Inhibitor is an antibody or antigen-binding antibody fragment (e.g., a single chain antibody, a single-domain antibody, a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor.

20. The method of any one of claims 1-19, wherein the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof.

21. The method of any one of claims 1-20, wherein the administered PFKFB3 Inhibitor is: (a) KAN0436151 or KAN0436067, or a salt thereof; (b) has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof; (c) has the structure of formula AZ44-AZ70 or AZ71, depicted in FIG. 1E, or a salt thereof; or (d) is AZ67, or a salt thereof.

22. The method of any one of claims 1-21, wherein the HIF1-α Pathway Inhibitor and the PFKFB3 inhibitor are co-administered to the subject.

23. The method of any one of claims 1-22, wherein the administration of the HIF1-α Pathway Inhibitor and/or the PFKFB3 inhibitor administration is oral, parenteral, orthotopic, intradermal, subcutaneous, intramuscular, intraperitoneal, intranasal, intratumoral, or intravenous.

24. The method of any one of claims 1-23, wherein the HIF1-α Pathway Inhibitor and the PFKFB3 inhibitor are administered before the onset of one or more symptoms of the pancreatic steatosis.

25. The method of any one of claims 1-24, wherein treating the pancreatic steatosis comprises delaying the onset of the pancreatic steatosis.

26. The method of any one of claims 1-23, wherein the HIF1-α Pathway Inhibitor and the PFKFB3 inhibitor are administered after the onset of one or more symptoms of the pancreatic steatosis.

27. The method of claim 26, wherein the method results in one or more symptoms of the pancreatic steatosis are reduced in the subject administered the HIF1-α Pathway Inhibitor and the PFKFB3 inhibitor compared to in the subject prior to treatment.

28. The method of claim 27, wherein the one or more reduced symptoms of the pancreatic steatosis is indicated by: a reduced fat content in the pancreas to less than 25%, reduced abdominal pain; reduced nausea, increased appetite, weight gain, reduced jaundice, reduced edema; and reduced tiredness, mental confusion, or weakness, reduced inflammation of the pancreas, reduced pancreatitis, reduced fibrosis in cells of the pancreas, and reduced levels of a pancreatic steatosis biomarker or pro-inflammatory cytokine (e.g., TNFα, IL-1β, IL6, MCP1, IL8, PAF).

29. The method of claim 27 or 28, wherein the one or more of the reduced symptoms is the reduction of fat content in the subject by at least 10%, at least 20%, at least 30%, at least 40%, or at least 50% compared to in the subject prior to treatment with the HIF1-α Pathway Inhibitor and the PFKFB3 inhibitor.

30. The method according to any one of claims 27-29, wherein the serum levels of at least 1, 2, 3, 4, or 5 pancreatic steatosis biomarkers is reduced by at least 20%, compared to in the subject prior to treatment with the HIF1-α Pathway Inhibitor and the PFKFB3 inhibitor.

31. The method according to any one of claims 27-29, wherein the serum levels of at least 1, 2, 3, 4, or 5 of the biomarkers: TNFα, IL-1β, IL6, MCP1, IL8, and/or PAF are reduced by at least 20%, compared to in the subject prior to treatment with the HIF1-α Pathway Inhibitor and the PFKFB3 inhibitor.

32. The method of any one of claims 1-31, which further comprises administering an additional therapeutic agent to the subject.

33. A method of treating an pancreatic cancer in a subject in need thereof comprising:
(a) administering an effective amount of a HIF1-α Pathway Inhibitor and an PFKFB3 inhibitor to the subject;
(b) administering an effective amount of a HIF1-α Pathway Inhibitor to the subject, wherein the subject has previously been administered a PFKFB3 Inhibitor; or
(c) administering an effective amount of a PFKFB3 Inhibitor to the subject, wherein the subject has previously been administered a HIF1-α Pathway Inhibitor;
wherein the PFKFB3 inhibitor does not inhibit PI3K/AKT/mTOR pathway or HIF1-α.

34. The method of claim 33, wherein the subject is administered an effective amount of the HIF1-α Pathway Inhibitor and the PFKFB3 inhibitor.

35. The method of claim 33, wherein the subject is administered an effective amount of the HIF1-α Pathway Inhibitor and wherein the subject has previously been administered the PFKFB3 Inhibitor.

36. The method of claim 33, wherein the subject is administered an effective amount of the PFKFB3 Inhibitor and wherein the subject has previously been administered the HIF1-α Pathway Inhibitor.

37. The method of any one of claims 33-36, wherein the method of any one of claim 33(a)-33(c) is administered as a prophylactic treatment for the pancreatic cancer.

38. The method of any one of claims 33-36, wherein the subject has or is at risk of having the pancreatic cancer.

39. The method of any one of claims 33-36, wherein the subject has or has been diagnosed as having the pancreatic cancer.

40. The method of any one of claims 33-39, wherein the pancreatic cancer is an exocrine pancreatic cancer or a neuroendocrine pancreatic cancer.

41. The method of claim 40, wherein the pancreatic cancer is an exocrine pancreatic cancer (e.g., an adenocarcinoma, squamous cell carcinoma, adenosquamous carcinoma, or a colloid carcinoma).

42. The method of claim 40, wherein the pancreatic cancer is a ductal adenocarcinoma (PDAC).

43. The method of claim 40, wherein the pancreatic cancer is a squamous cell carcinoma, adenosquamous carcinoma, or a colloid carcinoma.

44. The method of claim 40, wherein the pancreatic cancer is a neuroendocrine pancreatic cancer.

45. The method of any one of claims 33-44, wherein the administered HIF1-α Pathway Inhibitor is an antibody or antigen-binding antibody fragment (e.g., a single chain antibody, a single-domain antibody (e.g., a VHH), a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, dsRNA, ssRNA, and shRNA), a peptibody, a nanobody, a HIF1-α Pathway binding polypeptide, or a small molecule HIF1-α Pathway Inhibitor.

46. The method of any one of claims 33-45, wherein the administered HIF1-α Pathway Inhibitor is silibinin, PX-478 or YC-1, or a salt thereof.

47. The method of any one of claims 33-45, wherein the administered HIF1-α Pathway Inhibitor is ganetespib (ST-9090), phenethyl isothocyanate, or BAY-87-2243, or a salt thereof.

48. The method of any one of claims 33-47, wherein the administered HIF1-α Pathway Inhibitor is a HIF1-α Inhibitor.

49. The method of claim 48, wherein the HIF1-α Inhibitor is an antibody or antigen-binding antibody fragment (e.g., a single chain antibody, a single-domain antibody (e.g., a VHH), a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α binding polypeptide, or a small molecule HIF1-α Inhibitor.

50. The method of claim 48 or 49, wherein the administered HIF1-α Inhibitor is Antisense oligonucleotide EZN-2968 or nanobody AG-1, AG-2, AG-3, AG-4, AG-5, VHH212, or AHPC.

51. The method of any one of claims 33-50, wherein the administered PFKFB3 Inhibitor is an antibody or antigen-binding antibody fragment (e.g., a single chain antibody, a single-domain antibody, a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor.

52. The method of any one of claims 33-51, wherein the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof.

53. The method of any one of claims 33-51, wherein the administered PFKFB3 Inhibitor is: (a) KAN0436151 or KAN0436067, or a salt thereof; (b) has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof; (c) has the structure of formula AZ44-AZ70 or AZ71, depicted in FIG. 1E, or a salt thereof; or (d) is AZ67, or a salt thereof.

54. The method of any one of claims 33-53, wherein the HIF1-α Pathway Inhibitor and the PFKFB3 inhibitor are co-administered to the subject.

55. The method of any one of claims 33-54, wherein the administration of the HIF1-α Pathway Inhibitor and/or the PFKFB3 inhibitor administration is oral, parenteral, orthotopic, intradermal, subcutaneous, intramuscular, intraperitoneal, intranasal, intratumoral, or intravenous.

56. The method of any one of claims 33-55, wherein the HIF1-α Pathway Inhibitor and the PFKFB3 inhibitor are administered before the onset of one or more symptoms of the pancreatic cancer.

57. The method of any one of claims 33-56, wherein treating the pancreatic cancer comprises delaying the onset of the pancreatic cancer.

58. The method of any one of claims 33-57, wherein the HIF1-α Pathway Inhibitor and the PFKFB3 inhibitor are administered after the onset of one or more symptoms of the pancreatic cancer.

59. The method of any one of claims 33-58, wherein the method results in reduction in one or more symptoms of the pancreatic cancer in the subject compared to prior to administration of the HIF1-α Pathway Inhibitor and the PFKFB3 inhibitor.

60. The method of claim 59, wherein the one or more reduced symptoms of the pancreatic cancer is indicated by: decrease in tumor size, a suppression or decrease in tumor growth, no new tumor formation, a decrease in new tumor formation, an increase in survival or progression-free survival, no metastases, an increase in treatment options, delay in time from surgery to recurrence, reduction in jaundice, suppression of spread to liver, reduction in pain, improved appetite, improved digestion, reduction of gallbladder size, and reduced incidence of blood clots.

61. The method of claim 59 or 60, wherein the one or more symptoms of the pancreatic cancer are reduced by at least 10%, at least 20%, at least 30%, at least 40%, or at least 50% compared to in the subject prior to treatment with the HIF1-α Pathway Inhibitor and the PFKFB3 inhibitor.

62. The method according to any one of claims 59-61, wherein at least one of the biomarkers creatine kinase (CK-MB), troponin, N-terminal pro B-type natriuretic peptide, alpha-1 antitrypsin, C-reactive protein, apolipoprotein A1, apolipoprotein B, creatinine, alkaline phosphatase, and transferrin is reduced compared to in the subject prior to treatment with the HIF1-α Pathway Inhibitor and the PFKFB3 inhibitor.

63. The method according to any one of claims 59-62, wherein
(a) at least 1, 2, 3, 4, 5 or more of the biomarkers, creatine kinase (CK-MB), troponin, N-terminal pro B-type natriuretic peptide, alpha-1 antitrypsin, C-reactive protein, apolipoprotein A1, apolipoprotein B, creatinine, alkaline phosphatase, and transferrin of the subject are improved by at least 20%, at least 30%, at least 40%, or at least 50% compared to in the subject prior to treatment with the HIF1-α Pathway Inhibitor and the PFKFB3 inhibitor; or
(b) the tumor size in the subject is reduced by at least 20%, at least 30%, at least 40%, or at least 50% compared to in the subject prior to treatment with the HIF1-α Pathway Inhibitor and the PFKFB3 inhibitor

64. The method of any one of claims 33-63, which further comprises administering an additional therapeutic agent to the subject.

65. A method of treating non-alcoholic fatty liver disease (NAFLD) in a subject in need thereof comprising:
(a) administering an effective amount of a HIF1-α Pathway Inhibitor and an PFKFB3 inhibitor to the subject;
(b) administering an effective amount of a HIF1-α Pathway Inhibitor to the subject, wherein the subject has previously been administered a PFKFB3 Inhibitor; or
(c) administering an effective amount of a PFKFB3 Inhibitor to the subject, wherein the subject has previously been administered a HIF1-α Pathway Inhibitor;
wherein the PFKFB3 inhibitor does not inhibit PI3K/AKT/mTOR pathway or HIF1-α.

66. The method of claim 65, wherein the subject is administered an effective amount of the HIF1-α Pathway Inhibitor and the PFKFB3 inhibitor.

67. The method of claim 65, wherein the subject is administered an effective amount of the HIF1-α Pathway Inhibitor and wherein the subject has previously been administered the PFKFB3 Inhibitor.

68. The method of claim 65, wherein the subject is administered an effective amount of the PFKFB3 Inhibitor and wherein the subject has previously been administered the HIF1-α Pathway Inhibitor.

69. The method of any one of claims 65-68, wherein the method of any one of claim 65(a)-65(c) is administered as a prophylactic treatment for non-alcoholic fatty liver disease (e.g., non-alcoholic fatty liver (NAFL), non-alcoholic steatohepatitis (NASH), or NAFLD-associated liver fibrosis).

70. The method of any one of claims 65-68, wherein the subject has or is at risk of having non-alcoholic fatty liver disease (e.g., non-alcoholic fatty liver (NAFL), non-alcoholic steatohepatitis (NASH), or NAFLD-associated liver fibrosis).

71. The method of any one of claims 65-68, wherein the subject has or has been diagnosed as having non-alcoholic fatty liver disease (e.g., NAFL, NASH, or NAFLD-associated liver fibrosis).

72. The method of any one of claims 65-71, wherein the administered HIF1-α Pathway Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, dsRNA, ssRNA, and shRNA), a peptibody, a nanobody, a HIF1-α Pathway binding polypeptide, or a small molecule HIF1-α Pathway Inhibitor.

73. The method of any one of claims 65-72, wherein the administered HIF1-α Pathway Inhibitor is silibinin, PX-478 or YC-1, or a salt thereof.

74. The method of any one of claims 65-72, wherein the administered HIF1-α Pathway Inhibitor is ganetespib (ST-9090), phenethyl isothocyanate, or BAY-87-2243, or a salt thereof.

75. The method of any one of claims 65-72, wherein the administered HIF1-α Pathway Inhibitor is a HIF1-α Inhibitor.

76. The method of claim 75, wherein the HIF1-α Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α binding polypeptide, or a small molecule HIF1-α Inhibitor.

77. The method of claim 75 or 76, wherein the administered HIF1-α Inhibitor is Antisense oligonucleotide EZN-2968 or nanobody AG-1, AG-2, AG-3, AG-4, AG-5, VHH212, or AHPC.

78. The method of any one of claims 65-77, wherein the administered PFKFB3 Inhibitor is an antibody or antigen-binding antibody fragment (e.g., a single chain antibody, a single-domain antibody, a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor.

79. The method of any one of claims 65-78, wherein the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof.

80. The method of any one of claims 65-78, wherein the administered PFKFB3 Inhibitor is: (a) KAN0436151 or KAN0436067, or a salt thereof; (b) has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof; (c) has the structure of formula AZ44-AZ70 or AZ71, depicted in FIG. 1E, or a salt thereof; or (d) is AZ67, or a salt thereof.

81. The method of any one of claims 65-80, wherein the HIF1-α Pathway Inhibitor and the PFKFB3 inhibitor are co-administered to the subject.

82. The method of any one of claims 65-81, wherein the administration of the HIF1-α Pathway Inhibitor and/or the PFKFB3 inhibitor administration is oral, parenteral, orthotopic, intradermal, subcutaneous, intramuscular, intraperitoneal, intranasal, intratumoral, or intravenous.

83. The method of any one of claims 65-82, wherein the HIF1-α Pathway Inhibitor and the PFKFB3 inhibitor are administered before the onset of one or more symptoms of non-alcoholic fatty liver disease (e.g., non-alcoholic fatty liver (NAFL), non-alcoholic steatohepatitis (NASH), or NAFLD-associated liver fibrosis).

84. The method of any one of claims 65-83, wherein treating non-alcoholic fatty liver disease comprises delaying the onset of non-alcoholic fatty liver disease (e.g., non-alcoholic fatty liver (NAFL), non-alcoholic steatohepatitis (NASH), or NAFLD-associated liver fibrosis).

85. The method of any one of claims 65-84, wherein the HIF1-α Pathway Inhibitor and the PFKFB3 inhibitor are administered after the onset of one or more symptoms of non-alcoholic fatty liver disease (e.g., non-alcoholic fatty liver (NAFL), non-alcoholic steatohepatitis (NASH), or NAFLD-associated liver fibrosis).

86. The method of any one of claims 65-85, wherein the method results in reduction in one or more symptoms of non-alcoholic fatty liver disease in the subject compared to prior to administration of the HIF1-α Pathway Inhibitor and the PFKFB3 inhibitor.

87. The method of claim 86, wherein the one or more reduced symptoms of non-alcoholic fatty liver disease is indicated by:
(a) reduced fatigue, pain or discomfort in the upper right abdomen, reduction in spleen enlargement, reduced jaundice; reduced edema; increased appetite, and reduced;
(b) reduced NAS; or
(c) reduced levels reduced levels of NAFLD biomarkers (e.g., TNFα, IL-6, CRP, IL-1RA, PAI1, CXCL10, CK18, FGF21, oxLDL hyaluronic acid, laminin, procollagen II, and TIMP1).

88. The method of claim 86 or 87, wherein the one or more symptoms of non-alcoholic fatty liver disease are reduced by at least 10%, at least 20%, at least 30%, at least 40%, or at least 50% compared to in the subject prior to treatment with the HIF1-α Pathway Inhibitor and the PFKFB3 inhibitor.

89. The method according to any one of claims 86-88, wherein
(a) at least one of the plasma NAFLD biomarkers (e.g., TNFα, IL-6, CRP, IL-1RA, PAI1, CXCL10, CK18, FGF21, oxLDL, ALT, hyaluronic acid, laminin, procollagen II, and TIMP1) is improved compared to in the subject prior to treatment with the HIF1-α Pathway Inhibitor and the PFKFB3 inhibitor; or
(b) the NAS score in the subject is reduced by 1, 2, 3, or more compared to in the subject prior to treatment with the HIF1-α Pathway Inhibitor and the PFKFB3 inhibitor, or
(c) the treated subject has a NAS of <3 points.

90. The method according to any one of claims 86-89, wherein at least 1, 2, 3, 4, or 5, wherein at least 1, 2, 3, 4, or 5, biomarkers selected from TNFα, IL-6, CRP, IL-1RA, PAI1, CXCL10, CK18, FGF21, oxLDL hyaluronic acid, laminin, procollagen II, and TIMP1 are reduced by at least 20%, at least 30%, at least 40%, or at least 50% compared to in the subject prior to treatment with the HIF1-α Pathway Inhibitor and the PFKFB3 inhibitor.

91. The method of any one of claims 65-90, which further comprises administering an additional therapeutic agent to the subject.

92. A method of treating hepatocellular carcinoma in a subject in need thereof comprising:
(a) administering an effective amount of a HIF1-α Pathway Inhibitor and an PFKFB3 inhibitor to the subject;
(b) administering an effective amount of a HIF1-α Pathway Inhibitor to the subject, wherein the subject has previously been administered a PFKFB3 Inhibitor; or
(c) administering an effective amount of a PFKFB3 Inhibitor to the subject, wherein the subject has previously been administered a HIF1-α Pathway Inhibitor; and
wherein the PFKFB3 inhibitor does not inhibit PI3K/AKT/mTOR pathway or HIF1-α.

93. The method of claim 92, wherein the subject is administered an effective amount of the HIF1-α Pathway Inhibitor and the PFKFB3 inhibitor.

94. The method of claim 92, wherein the subject is administered an effective amount of the HIF1-α Pathway Inhibitor and wherein the subject has previously been administered the PFKFB3 Inhibitor.

95. The method of claim 92, wherein the subject is administered an effective amount of the PFKFB3 Inhibitor and wherein the subject has previously been administered the HIF1-α Pathway Inhibitor.

96. The method of any one of claims 92-95, wherein the method of any one of claim 92(a)-92(c) is administered as a prophylactic treatment for hepatocellular carcinoma.

97. The method of any one of claims 92-95, wherein the subject has or is at risk of having hepatocellular carcinoma.

98. The method of any one of claims 92-95, wherein the subject has or has been diagnosed as having hepatocellular carcinoma.

99. The method of any one of claims 92-98, wherein the administered HIF1-α Pathway Inhibitor is an antibody or antigen-binding antibody fragment (e.g., a single chain antibody, a single-domain antibody (e.g., a VHH), a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, dsRNA, ssRNA, and shRNA), a peptibody, a nanobody, a HIF1-α Pathway binding polypeptide, or a small molecule HIF1-α Pathway Inhibitor.

100. The method of any one of claims 92-99, wherein the administered HIF1-α Pathway Inhibitor is silibinin, PX-478 or YC-1, or a salt thereof.

101. The method of any one of claims 92-99, wherein the administered HIF1-α Pathway Inhibitor is ganetespib (ST-9090), phenethyl isothocyanate, or BAY-87-2243, or a salt thereof.

102. The method of any one of claims 92-99, wherein the administered HIF1-α Pathway Inhibitor is a HIF1-α Inhibitor.

103. The method of claim 102, wherein the HIF1-α Inhibitor is an antibody or antigen-binding antibody fragment (e.g., a single chain antibody, a single-domain antibody (e.g., a VHH), a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α binding polypeptide, or a small molecule HIF1-α Inhibitor.

104. The method of claim 102 or 103, wherein the administered HIF1-α Inhibitor is Antisense oligonucleotide EZN-2968 or nanobody AG-1, AG-2, AG-3, AG-4, AG-5, VHH212, or AHPC.

105. The method of any one of claims 92-104, wherein the administered PFKFB3 Inhibitor is an antibody or antigen-binding antibody fragment (e.g., a single chain antibody, a single-domain antibody, a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor.

106. The method of any one of claims 92-105, wherein the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof.

107. The method of any one of claims 92-105, wherein the administered PFKFB3 Inhibitor is: (a) KAN0436151 or KAN0436067, or a salt thereof; (b) has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof; (c) has the structure of formula AZ44-AZ70 or AZ71, depicted in FIG. 1E, or a salt thereof; or (d) is AZ67, or a salt thereof.

108. The method of any one of claims 92-107, wherein the HIF1-α Pathway Inhibitor and the PFKFB3 inhibitor are co-administered to the subject.

109. The method of any one of claims 92-108, wherein the administration of the HIF1-α Pathway Inhibitor and/or the PFKFB3 inhibitor administration is oral, parenteral, orthotopic, intradermal, subcutaneous, intramuscular, intraperitoneal, intranasal, intratumoral, or intravenous.

110. The method of any one of claims 92-109, wherein the HIF1-α Pathway Inhibitor and the PFKFB3 inhibitor are administered before the onset of one or more symptoms of hepatocellular carcinoma.

111. The method of any one of claims 92-110, wherein treating hepatocellular carcinoma comprises delaying the onset of hepatocellular carcinoma.

112. The method of any one of claims 92-111, wherein the HIF1-α Pathway Inhibitor and the PFKFB3 inhibitor are administered after the onset of one or more symptoms of hepatocellular carcinoma.

113. The method of any one of claims 92-112, wherein the method results in reduction in one or more symptoms of hepatocellular carcinoma the subject administered the HIF1-α Pathway Inhibitor and the PFKFB3 inhibitor compared to in the subject prior to treatment.

114. The method of claim 113, wherein the one or more reduced symptoms of hepatocellular carcinoma is indicated by:
(a) weight gain, increased appetite, reduced fever, reduced nausea, reduced fatigue, reduced upper abdominal pain, reduced swelling in the abdomen and legs, reduced yellowing of the skin (jaundice), or reduced bruising;
(b) reduction of at least one biomarker typically elevated in subjects having HCC (e.g., EpCam, VEGF, EGFR, FLT1, theophylline, HCC-22-5, KRT23, AHSG, FTL, C16Cer, C16DHC, C18DHC, S1P, C24DHC, C24:1DHC, C18Cer, C20Cer, C24Cer, C24:1Cer, Sphingosine, and SA1P, CTSD, HYOU1, PSAP, and LAMP-2); or
(c) a decrease in tumor size, a suppression or decrease in tumor growth, no new tumor formation, a decrease in new tumor formation, an increase in survival or progression-free survival, no metastases, an increase in treatment options, or a delay in time from surgery to recurrence.

115. The method of claim 113 or 114, wherein at least one of the following symptoms is improved in the subject compared to prior to treatment with the HIF1-α Pathway Inhibitor and the PFKFB3 inhibitor: weight loss, loss of appetite, fever, nausea, fatigue, upper abdominal pain, swelling in the abdomen and legs, yellowing of the skin (jaundice), easy bruising or bleeding, and liver failure.

116. The method any one of claims 113-115, wherein at least 1, 2, 3, 4, or 5 biomarkers selected from: EpCam, VEGF, EGFR, FLT1, theophylline, HCC-22-5, KRT23, AHSG, FTL, C16Cer, C16DHC, C18DHC, S1P, C24DHC, C24:1DHC, C18Cer, C20Cer, C24Cer, C24:1Cer, Sphingosine, and SA1P, CTSD, HYOU1, PSAP, and LAMP-2, is reduced at least 10%, at least 20%, at least 30%, at least 40%, or at least 50% compared to in the subject prior to treatment with the HIF1-α Pathway Inhibitor and the PFKFB3.

117. The method according to any one of claims 113-116, wherein the tumor size in the subject is reduced by at least 20%, at least 30%, at least 40%, or at least 50% compared to in the subject prior to treatment with the HIF1-α Pathway Inhibitor and the PFKFB3 inhibitor.

118. The method of any one of claims 92-117, which further comprises administering an additional therapeutic agent to the subject.

119. The method of any one of claims 1-118, which further comprises administering a GPR81 Inhibitor to the subject.
